(19) United States
(12) Patent Application Publication
(10) Pub. No.: US 2023/0069897 A1
(43) Pub. Date: Mar. 9, 2023

(54) APPARATUS FOR STORING AN ORGAN OR BODY TISSUE, AND CORRESPONDING METHOD

(71) Applicant: UNIVERSITY OF NEWCASTLE UPON TYNE, Newcastle upon Tyne (GB)

(72) Inventors: William Earl SCOTT, III, Newcastle upon Tyne (GB); Stuart Brian William KAY, Newcastle upon Tyne (GB)

(21) Appl. No.: 17/798,962

(22) PCT Filed: Feb. 12, 2021
(86) PCT No.: PCT/GB2021/050351

§ 371 Date: Aug. 11, 2022

(30) Foreign Application Priority Data
Feb. 12, 2020 (GB) 2001924.6
Feb. 12, 2020 (GB) 2001925.3

(51) Int. Cl. A01N 1/02; A61B 46/00

(52) U.S. Cl. CPC A01N 1/0247; A01N 1/0273; A61B 46/40

(57) ABSTRACT

An apparatus for storing an organ or body tissue is disclosed, comprising: a container unit comprising a container body defining a storage region, wherein the container body includes an opening to enable the organ or body tissue to be admitted to, and removed from, the storage region; and a surgical drape (802) comprising: a securing portion (804) which is securable to the container unit, a peripheral outer portion (808) which surrounds the securing portion, and a frangible portion (810) separating the securing portion from the peripheral outer portion; wherein the securing portion is securable to the container body to enable the drape to extend around a portion of the periphery of the opening with the peripheral outer portion of the drape arranged outward of the opening; and wherein the frangible portion extends around the securing portion to enable the peripheral outer portion to be separated from the securing portion by tearing the frangible portion.

### TECHNICAL FIELD

The present disclosure relates to apparatuses and methods for storing an organ or body tissue, such as for organ transplant surgery. This may include apparatuses and methods for preservation and/or transport of the organ or body tissue.

### BACKGROUND

Gaseous oxygen perfusion or ‘Persufflation’ (PSF) is a technique for delivering oxygen via the native vasculature with the goal of maintaining the bio-energetic status of an organ or tissue. There are two main approaches to persufflation: anterograde persufflation (A-PSF), a physiological flow of gas entering through the artery and draining through the vein; and retrograde persufflation (R-PSF), where gas enters through the vein and exits through holes pricked into the surface of the organ or tissue with a needle.

US patent applications US2010/0330547 A1 and US 20120178150 A1 relate to perfusion devices based on electrochemical gas generation. Such devices are typically used in a clinical environment for the temporary storage of an organ or body tissue.

### SUMMARY

Aspects of the disclosure are set out in the independent claims and optional features are set out in the dependent claims. Aspects of the disclosure may be provided in conjunction with each other, and features of one aspect may be applied to other aspects. Aspects described herein generally relate to each of: (i) a container unit, (ii) a storage apparatus, (iii) a drape, and (iv) an automated method. These aspects, and their features disclosed herein, may be combined with each of the other aspects. For example, one apparatus or method may be defined which includes features of two or more of these aspects. The possibility of combining these different aspects will be apparent from the following description.

In an aspect, there is provided a container unit for storing an organ or body tissue, wherein the container unit is configured as an insert for a storage apparatus for preserving a said organ or body tissue. The container unit comprises: (i) a container body defining a storage region for storing an organ or body tissue; (ii) an inlet port for connection to a fluid supply system of a said storage apparatus to receive persufflation fluid from the storage apparatus; (iii) an outlet port for connection to a said organ or body tissue stored in the container body to enable persufflation fluid to be delivered to said organ or body tissue; and (iv) a fluid processing device comprising an internal passageway connecting the inlet port to the outlet port to enable persufflation fluid to flow from the inlet port to the outlet port, wherein the fluid processing device is configured to process persufflation fluid flowing through the internal passageway from the inlet port to the outlet port.

Aspects may enable the provision of a disposable container unit for storing an organ or body tissue, e.g. a single-use container unit. This may have an advantage of enabling the unit to be maintained sterile, as it is only used for one organ. It may have an advantage of enabling a storage apparatus to be provided which may be used a plurality of times (each time with a different container unit) without requiring substantial maintenance (such as cleaning of the storage apparatus, e.g. without excessive cleaning, or cleaning and sterilizing).

The fluid supply system of the storage apparatus may comprise an oxygen supply system and the persufflation fluid may comprise a gaseous mixture. The inlet port may be for connection to a said oxygen supply system of a said storage apparatus to receive a gaseous mixture therefrom. The fluid processing device may comprise a gas humidifier and heat exchanger. The storage region may be arranged to receive an organ preservation liquid in which the organ or body tissue is to be stored. The internal passageway may comprise an organ preservation liquid inlet arranged to enable organ preservation liquid in the storage region to flow into the internal passageway. At least one surface of the internal passageway may be arranged to provide heat exchange between organ preservation liquid in the storage region and gaseous mixture in the internal passageway. The fluid processing device being configured to process persufflation fluid flowing through the internal passageway from the inlet port to outlet port may comprise the gas humidifier and heat exchanger being configured to provide humidifying and cooling of the gaseous mixture prior to supplying said cooled and humidified gaseous mixture to said organ or body tissue.

For example, aspects may provide a container unit for storing an organ or body tissue, wherein the container unit is configured as an insert for a storage apparatus for preserving a said organ or body tissue, wherein the container unit comprises: a container body defining a storage region for storing an organ or body tissue, wherein the storage region is arranged to receive an organ preservation liquid in which the organ or body tissue is to be stored; an inlet port for connection to an oxygen supply system of a said storage apparatus to receive a gaseous mixture from the storage apparatus; an outlet port for connection to a said organ or body tissue stored in the container body to enable the gaseous mixture to be delivered to said organ or body tissue; and a gas humidifier and heat exchanger comprising an internal passageway connecting the inlet port to the outlet port to enable the gaseous mixture to flow from the inlet port to the outlet port, wherein: the internal passageway comprises an organ preservation liquid inlet arranged to enable organ preservation liquid in the storage region to flow into the internal passageway; and at least one surface of the internal passageway is arranged to provide heat exchange between organ preservation liquid in the storage region and gaseous mixture in the internal passageway; to provide humidifying and cooling of the gaseous mixture prior to supplying said cooled and humidified gaseous mixture to said organ or body tissue.

Being configured as an insert may comprise being sized and/or shaped to fit within a container unit receiving portion of a storage apparatus. It may comprise the container unit comprising one or more attachment portion for securing the container unit to the storage apparatus, such as a mechanical connection e.g. a clip. The insert may be removably inserted within the storage apparatus (e.g. arranged to be both placed in and taken out of the storage apparatus). The storage region may provide a region in which preservation fluid may be placed to enable the organ or body tissue to rest in/on the preservation fluid within the storage region. The container unit may comprise a single unit, such as a single integrated unit. The single unit may be movable independently of the storage apparatus.

The inlet port may comprise a hole to which a tube (such as a fluid flow line) from the storage apparatus may be connected (e.g. inserted through). The outlet port may comprise an attachment for connection to an additional device. For example the outlet port may be connected to a cannulae, catheter or other connector for insertion within the organ or tissue. The fluid processing device may be configured to alter at least one property of fluid flowing through the internal passageway. For example, this property may be at least one of: (i) temperature, (ii) humidity, (iii) bubble size, (iv) pressure, (v) flow rate, and (vi) persufflation fluid concentration. The fluid processing device may be configured so that the at least one property of the fluid which is altered is altered by the flow of the fluid through the internal passageway. For example, the internal passageway may be arranged so that the flow of fluid through the internal passageway alters the at least one property of the fluid.

The fluid processing device may comprise at least one obstruction arranged within the internal passageway to disrupt the flow of persufflation fluid flowing through the internal passageway from the inlet port to the outlet port. For example, the cross-section (and/or the cross-sectional area) and/or orientation of the internal passageway may change along its length. The internal passageway may be arranged to provide some disruption to fluid flowing through it, e.g. by the inclusion of an obstacle in the way of the fluid flow. The at least one obstruction may comprise at least one bubble break element arranged within the internal passageway to reduce a bubble size of the persufflation fluid flowing through the internal passageway from the inlet port to the outlet port. A bubble break element may comprise an element which is sized and/or shaped so that bubbles in fluid (e.g. gas) flowing past the element will be reduced in size. This may comprise use of elements which protrude into the internal passageway, e.g. which are small and/or angular enough in one region of the element to reduce bubble size. The fluid processing device may be configured to process persufflation fluid to be in an improved (e.g. more suitable) state for persufflating an organ or tissue. The fluid processing device may be configured to improve fluid properties prior to insertion of the fluid into the organ or tissue. For example, the fluid processing device may be configured to alter properties of the persufflation fluid to reduce the likelihood of that fluid damaging the tissue or organ.

The internal passageway may be arranged to define at least one bend through which the persufflation fluid will flow when flowing from the inlet port to the outlet port. For example, this may include a change in direction of the fluid flow, as it flows from the inlet towards the outlet through the internal passageway. The bend may be greater than 90 degrees, such as a 180 degree bend. The internal passageway may follow a curved, e.g. tortuous path. The path of the internal passageway may be shaped turbulent flow of fluid, e.g. with flow going round the bend. The path may follow at least one 180 degree bend, e.g. to enable the flow path to travel into a region in which preservation fluid from the storage region is present and back into a region where it is not (e.g. where the outlet is located). The passageway may include at least two stretches which are parallel to one another (e.g. to enable more volume-efficient use of a longer internal passageway). The fluid processing device may be arranged to provide a serpentine shape to the internal passageway. For example, the internal passageway may have a serpentine shape.

The fluid processing device may comprise a preservation fluid inlet arranged to enable preservation fluid in the storage region to flow into the internal passageway. Preservation fluid may be able to flow into a region of the internal passageway. For example, the persufflation fluid flowing through the internal passageway may flow from the inlet to the outlet, while flowing through a region of the internal passageway in which there is preservation fluid (e.g. the persufflation fluid may flow through the preservation fluid). The fluid processing device may further comprise a one-way valve arranged to inhibit flow of persufflation fluid from the internal passageway through the preservation fluid inlet into the storage region. For example, preservation fluid may be able to flow from the storage region into the internal passageway through the preservation fluid inlet, but persufflation fluid may be unable to flow from the internal passageway through the preservation fluid inlet and into the storage region.

The fluid processing device may comprise a heat exchanger arranged to provide heating and/or cooling of persufflation fluid in the internal passageway. The heat exchanger may comprise an element arranged to alter temperature of the persufflation fluid in the internal passageway. This may be active or passive heating. The heat exchanger may be arranged to redistribute heat from different regions of the container unit. The heat exchanger may be arranged to exchange heat energy between persufflation fluid in the internal passageway and preservation fluid in the storage region. For example if one of the fluids is colder than the other, then this arrangement may enable cooling of the respective other fluid, e.g. to provide two fluids of similar temperatures. The container unit may be arranged to have at least one surface of the fluid processing device in contact with preservation fluid in the storage region. For example, the fluid processing device may extend from a wall of the container unit inwards towards the centre of the container unit, e.g. into a region where the organ or tissue is stored and e.g. where the preservation fluid is.

The fluid processing device may be arranged to humidify persufflation fluid flowing through the internal passageway. For example, the fluid processing device may be configured to enable condensation within the internal passageway, e.g. from water contained within the air or the preservation fluid. At least one surface of the fluid processing device may be integral with the storage region. For example, preservation fluid stored in the storage region may be in contact with at least one surface of the fluid processing device.

The container unit may comprise a first venting port operable to enable pressure within the container unit and/or storage region to be reduced. For example, venting ports may enable gases within the container unit to flow out through the venting port (e.g. to the atmosphere). The venting port may comprise an element which may be user actuated to enable venting, and/or a pressure relief valve may be used, e.g. so that when pressure gets above a threshold level, gas is vented. The container unit may comprise an outer lid for sealing the container unit. This may provide a hermetic seal, e.g. to inhibit external contaminants from the atmosphere coming into contact with the organ or tissue contained within the container unit. The outer lid may comprise the first venting port, e.g. the first venting port may be provided within the outer lid. The container unit may comprise an inner lid arranged to seal the organ or body tissue within the storage region. For example, the inner lid may provide a hermetic seal (e.g. to decrease the likelihood of environmental contaminants coming into contact with the organ or tissue). The inner lid may provide some securing of the organ or tissue within the storage region. The inner lid may comprise a second venting port operable to enable pressure within the storage region to be reduced.

Each obstruction may comprise a baffle. Each baffle may extend crosswise with respect to the internal passageway and comprises at least one of: (i) a pin, (ii) a sphere and (iii) an ellipsoid. For example, the baffle may include an element which extends from an outer surface defining the internal passageway into the internal passageway itself. Each pin may have at least one of: (i) a circular cross-section, (ii) an ellipsoid cross-section, and (iii) a polygonal cross-section. The storage region may be arranged to hold preservation fluid to enable the organ or body tissue to be stored together with the preservation fluid. The storage region may be arranged to enable the outlet port to be above a level of the preservation fluid within the storage region.

In an aspect there is provided a storage apparatus including a container unit disclosed herein. For example, in an aspect, there is provided a storage apparatus for preserving an organ or body tissue, the apparatus comprising: a fluid supply system comprising a store of persufflation fluid and a flow line which connects the store of persufflation fluid to a fluid supply outlet port to enable persufflation fluid from the store to flow to the fluid supply outlet port; a container unit receiving portion; and a container unit which is removably insertable into the container unit receiving portion, wherein the container unit comprises: (i) a container body defining a storage region for storing a said organ or body tissue, (ii) an inlet port for connection to the fluid supply outlet port, (iii) an outlet port for connection to a said organ or body tissue stored in the container body to enable persufflation fluid to be delivered to said organ or body tissue, and (iv) a fluid processing device comprising an internal passageway connecting the inlet port to the outlet port to enable persufflation fluid to flow from the inlet port to the outlet port, wherein the fluid processing device is configured to process persufflation fluid flowing through the internal passageway from the inlet port to the outlet port. In use for preserving an organ or body tissue, the container unit is inserted into the container unit receiving portion, and the inlet port of the container unit is connected to the fluid supply outlet port to persufflation fluid from the store of persufflation fluid to flow through the flow line into the internal passageway of the fluid processing device and into the organ or body tissue stored in the storage region.

In an aspect, there is provided a kit of parts comprising: a storage apparatus for preserving an organ or body tissue; a store of persufflation fluid; and a container unit as disclosed herein.

In an aspect, there is provided a method of preparing an apparatus to store and preserve an organ or body tissue. The method comprising: inserting a container unit into a storage apparatus, wherein the container unit comprises: (i) a container body defining a storage region for storing a said organ or body tissue; (ii) an inlet port for connection to a fluid supply system of the storage apparatus to receive persufflation fluid from the storage apparatus; (iii) an outlet port for connection to a said organ or body tissue stored in the container body to enable persufflation fluid to be delivered to said organ or body tissue; and (iv) a fluid processing device comprising an internal passageway connecting the inlet port to the outlet port to enable persufflation fluid to flow from the inlet port to the outlet port, wherein the fluid processing device is configured to process persufflation fluid flowing through the internal passageway from the inlet port to the outlet port, and wherein the storage apparatus comprises a fluid supply system comprising a store of persufflation fluid and a flow line which connects the store of persufflation fluid to a fluid supply outlet port to enable persufflation fluid from the store to flow to the fluid supply outlet port; connecting the fluid supply outlet port of the storage apparatus to the inlet port of the container unit to enable persufflation fluid to flow from the store of persufflation fluid in the storage apparatus through to the organ or tissue stored in the storage region of the container unit. For example, method aspects may comprise insertion of a container unit, as disclosed herein, into a storage apparatus, as disclosed herein, and connecting the two to enable persufflation fluid stored in the storage apparatus to be applied to an organ or body tissue stored in the container unit.

In an aspect, there is provided an apparatus for storing an organ or body tissue, the apparatus comprising: a container unit comprising a container body defining a storage region for storing an organ or body tissue, wherein the container body includes an opening to enable the organ or body tissue to be admitted to, and removed from, the storage region through the opening; and a surgical drape comprising: (i) a securing portion which is securable to the container unit, (ii) a peripheral outer portion which surrounds the securing portion, and (iii) a frangible portion separating the securing portion from the peripheral outer portion. The securing portion is securable to the container body to enable the drape to extend around a portion of the periphery of the opening with the peripheral outer portion of the drape arranged outward of the opening. The frangible portion extends around the securing portion to enable the peripheral outer portion to be separated from the securing portion by tearing the frangible portion.

Aspects may enable a sterile environment to be maintained in areas surrounding the organ. The drape may be used to maintain sterility of a surface beneath it (and/or to reduce the likelihood of contaminants associated with that surface coming into contact with the organ). Then, once the organ has been placed in the storage region of the container unit, the outer portion of the drape may be removed. This may inhibit the likelihood of the outer portion of the drape interfering with other parts of the storage apparatus. The outer portion of the drape may then be discarded, and the organ stored and/or transported when in the container unit (e.g. as inserted into a storage apparatus). The securing portion of the surgical drape may be secured to the container unit. A storage apparatus may be provided which comprises: (i) a fluid supply system comprising a store of persufflation fluid to be supplied to a said organ or body tissue, and (ii) a container unit receiving portion, wherein the container unit is removably insertable into the container unit receiving portion. A kit may be provided for the container unit and drape, and e.g. for the storage apparatus as well.

The container unit may be arranged so that the storage region defines a volume in which preservation fluid may be stored. A wall may extend around the periphery of the storage region (e.g. which defines the volume in which preservation fluid may be stored). This wall may also define the opening in the container unit. The securing portion of the drape may comprise a region of the drape which is adapted to be secured (e.g. attached) to a corresponding region of the container unit. The securing portion may comprise one or more attachment means to facilitate attachment to the container unit, e.g. the securing portion may comprise an adhesive. The securing portion may be sized to correspond to a corresponding attachment portion of the container unit. The frangible portion may extend circumferentially around the securing portion. Tearing the frangible portion may comprise tearing along the frangible portion. For example, it may comprise applying a force to the drape (e.g. by pulling the peripheral outer portion) which causes the frangible portion to tear away.

The drape may be circular (e.g. annular with an access opening in the middle), or it may take other shapes. Working radially outwards from the centre of the drape, the securing portion may be located radially inwards from the peripheral outer portion, e.g. with the frangible portion radially between these two. The frangible portion may have a weakness defined therein to facilitate easy tearing. For example, it may include a plurality of cuts where material has been removed, or where the material has been scored (e.g. by knife or laser). The drape may be arranged so that when installed on the container unit (with the securing portion secured to the container unit) the peripheral outer portion extends away from the opening. It is to be appreciated that the exact location of the securing portion, frangible portion and peripheral outer portion need not be considered limiting, as long as at least a portion of the peripheral outer portion is provided outwards from the opening to provide some protection. For example, a portion of the securing portion may also extend away from the opening, or alternatively both the frangible portion and the securing portion may be in contact with the container body (a portion of the peripheral outer portion may also be in contact with the container body).

The container unit may have an attachment portion to which the surgical drape is securable. The attachment comprise may comprise a wall, e.g. which extends around a region of the container unit to define the storage region and/or the opening. The attachment portion may surround at least a portion of the opening of the container unit. The securing portion of the drape may comprise an adhesive region for securing the drape to the container unit. The drape may include an access opening which corresponds to the opening of the container unit to enable surgical drape to be secured to the container unit with the access opening in alignment with the opening of the container unit. The access opening may comprise a hole of the same size as, or larger than, the size of the opening hole. The access opening may be arranged to enable the drape to be placed over the container unit with the opening being exposed through the access opening (e.g. to enable a user to place the organ through both openings into the storage region of the container unit). The adhesive region may surround at least a portion of the access opening. The drape may be arranged to surround the opening, such as by circumscribing, but not necessarily completely circumscribing, the opening. At least a portion of the peripheral outer portion may be arranged outward of the opening (e.g. radially outward) to provide protection for a surface underneath that portion of the drape. For example, the peripheral outer portion may be arranged about the opening.

The surgical drape may comprise a first surgical drape. The apparatus may further comprise a second surgical drape (e.g. the apparatus may include two separate drapes). The second surgical drape may comprise a second securing portion which is securable to the container body to enable the second drape to extend around a portion of the periphery of the opening. Both drapes may be attached to the container unit, e.g. to different parts of the container unit. The container unit may comprise a second attachment portion to which the second surgical drape is securable. The second attachment portion may be different to the first portion. The second attachment portion may comprise an inner surface of a wall of the container body. For example, a wall may extend around the storage region and/or the opening, and the second attachment portion may comprise a portion of that wall, such as on the inside (e.g. radially) of the wall.

The container unit may further comprise an inner lid member which is configured to be secured to the container body so as to enclose the organ or body tissue within the storage region between the container body and the inner lid member. The second attachment portion may be an inner surface of a wall of a container unit which is external the storage region enclosed by the lid member and the container body. The apparatus may be arranged to enable the second drape to be secured to the container body so that a portion of the second drape overlies the inner lid member. The container unit may comprise an outer lid member which is configured to be secured to the container body so as to enclose the inner lid member between the container body and the outer lid member.

In an aspect, there is provided a surgical drape configured to be secured to a container unit for storing an organ or body tissue, the surgical drape comprising: an access opening arranged to enable an organ or body tissue to be passed through the access opening; a securing portion surrounding the access opening which is securable to a securing portion of a said container unit; a peripheral outer portion which surrounds the securing portion; and a frangible portion separating the securing portion from the peripheral outer portion. The securing portion is securable to the container body to: (i) enable the access opening of the drape to align with an opening of the container body, (ii) enable the drape to extend around a portion of the periphery of the opening of the container unit with the peripheral outer portion of the drape arranged outward of the opening. The frangible portion extends around the securing portion to enable the peripheral outer portion to be separated from the securing portion by tearing the frangible portion. The securing portion of the drape may comprise an adhesive region for securing the drape to the container unit.

In an aspect, there is provided an apparatus for preserving an organ or body tissue, the apparatus comprising: a storage apparatus comprising: (i) a fluid supply system comprising a store of persufflation fluid to be supplied to a said organ or body tissue, and (ii) a container unit receiving portion; a container unit removably insertable into the container unit receiving portion, wherein the container unit comprises a container body defining a storage region for storing an organ or body tissue, wherein the container body includes an opening to enable the organ or body tissue to be admitted to, and removed from, the storage region through the opening; and a surgical drape comprising: (i) a securing portion which is securable to the container unit, (ii) a peripheral outer portion which surrounds the securing portion, and (iii) a frangible portion separating the securing portion from the peripheral outer portion. The securing portion of the drape is securable to the container body to enable the drape to extend around a portion of the periphery of the opening with the peripheral outer portion of the drape arranged outward of the opening to protect a portion of a surface of the storage apparatus. The frangible portion extends around the securing portion to enable the peripheral outer portion to be separated from the securing portion by tearing the frangible portion. The portion of the surface of the storage apparatus may comprise a portion of a periphery of the container unit receiving portion.

It will be appreciated in the context of the present disclosure that apparatuses for storing an organ or body tissue disclosed herein may be provided assembled or unassembled. For example, different components of the apparatus may be attached to other components within the apparatus, or they may be arranged to be attached, but are not provided attached. In an aspect, there is provided a kit of parts comprising a container unit as disclosed herein and a surgical drape as disclosed herein. The kit may further comprise a storage apparatus as disclosed herein.

In an aspect, there is provided an apparatus for storing an organ or body tissue, the apparatus comprising: a container unit comprising a container body defining a storage region for storing an organ or body tissue, wherein the container body includes an opening to enable the organ or body tissue to be admitted to, and removed from, the storage region through the opening; and a surgical drape comprising: (i) a securing portion secured to the container unit, (ii) a peripheral outer portion which surrounds the securing portion, and (iii) a frangible portion separating the securing portion from the peripheral outer portion. The securing portion is secured to the container body so that the drape extends around a portion of the periphery of the opening with the peripheral outer portion of the drape arranged outward of the opening. The frangible portion extends around the securing portion to enable the peripheral outer portion to be separated from the securing portion by tearing the frangible portion.

In an aspect, there is provided an apparatus for preserving an organ or body tissue, the apparatus comprising: a storage apparatus comprising: (i) a fluid supply system comprising a store of persufflation fluid to be supplied to a said organ or body tissue, and (ii) a container unit receiving portion; a container unit removably insertable into the container unit receiving portion, wherein the container unit comprises a container body defining a storage region for storing an organ or body tissue, wherein the container body includes an opening to enable the organ or body tissue to be admitted to, and removed from, the storage region through the opening; and a surgical drape comprising: (i) a securing portion which is secured to the container unit, (ii) a peripheral outer portion which surrounds the securing portion, and (iii) a frangible portion separating the securing portion from the peripheral outer portion; wherein the securing portion of the drape is secured to the container body so that the drape extends around a portion of the periphery of the opening with the peripheral outer portion of the drape arranged outward of the opening to protect a portion of a surface of the storage apparatus; and wherein the frangible portion extends around the securing portion to enable the peripheral outer portion to be separated from the securing portion by tearing the frangible portion.

In an aspect, there is provided a method of providing an organ or body tissue in the apparatuses for storing an organ or body tissue disclosed herein. The method comprises: placing the organ or body tissue through the opening in the container unit and into the storage region of the container unit; and tearing along the frangible portion of the drape to remove the peripheral outer portion of the drape after the organ or body tissue has been placed into the storage region of the container unit. The method may comprise attaching an inner lid member to the storage region of the container unit. It may comprise attaching a second drape so that a portion of the second drape overlies the inner lid member. The method may comprise removing the organ or body tissue from the storage region after tearing the frangible portion.

In an aspect, there is provided an apparatus for storing an organ or body tissue. The apparatus comprises: a container unit having a container body which defines a storage region for storing an organ or body tissue, wherein the size and/or shape of the storage region is configured for storing a predetermined type of organ or body tissue; and a base unit configured to removably receive the container unit, the base unit comprising a fluid supply system arranged to supply a persufflation fluid to an organ or body tissue stored within the storage region when the container unit is received within the base unit.

The container unit may have at least one inlet port in fluid communication with at least one outlet port, wherein the inlet port is detachably connectable to the fluid supply system and the outlet port is connectable to a device for delivering persufflation fluid to an organ or body tissue when stored in the storage region. The storage unit (e.g. base unit) may be configured to maintain the temperature of an organ stored or body tissue in the container unit within a predetermined temperature range, for example between 4 degrees centigrade and 25 degrees centigrade. The base unit may comprise a temperature regulating element for regulating the temperature of the organ or body tissue in the container unit. The temperature regulating element may comprise at least one of a cooling element and a heating element. The temperature regulating element may be active or passive.

The base unit may comprise a docking portion configured to receive the container unit. The base unit may comprise a thermally insulating material arranged around at least a portion of the periphery of the docking portion to form an insulating layer. The thermally insulating material may have an R-value of not less than18 RSI per metre (m·K/W). The thermally insulating material may comprise at least to one of closed-cell polyurethane spray foam, moulded expanded polystyrene (EPS) low-density, moulded expanded polystyrene (EPS) high-density, cardboard or vermiculite. The base unit may comprise at least one compartment for receiving a cooling or heating element. The base unit may comprise a power source and a control system configured to control the persufflation fluid supply system. At least one of the power source and the control system may be disposed externally with respect to the docking portion and the insulating layer.

The persufflation fluid supply system may be configured to supply a gas comprising at least 20% oxygen, for example at least 40% oxygen, for example at least 95% oxygen. The fluid supply system may comprise a gas source for delivering a persufflating fluid comprising at least 20% oxygen, for example at least 40% oxygen, for example at least 95% oxygen. The gas source may be disposed externally with respect to the insulating layer. The gas source may comprise a source of compressed gas. The fluid supply system may comprise a plurality of fluid channels arranged to persufflate different portions of the organ or body tissue. The apparatus may be for anterograde persufflation of a pancreas for islet isolation. The base unit may comprise a lower portion and a lid member, wherein the lower portion is configured to removably receive the container unit and the lid member is configured to secure the container unit within the base unit. The container unit may have a storing portion and a container unit lid member which is removably securable to the storing portion in order to seal the storage region. The apparatus may be portable, mobile or handheld.

In an aspect, there is provided a base unit configured to removably receive different container units, each container unit having a container body which is configured to be received by the base unit and which defines a storage region having a size and/or shape configured for storing a predetermined type of organ or body tissue, the base unit comprising a fluid supply system arranged to supply a persufflation fluid to an organ received within the container unit when the container unit is disposed within the base unit.

In an aspect, there is provided a container unit having a container body which defines a storage region for storing an organ or body tissue, wherein the size and/or shape of the storage region is configured for storing a predetermined type of organ or body tissue and the container unit is configured to be removably received within the base unit of storage apparatuses disclosed herein. The container unit may have at least one inlet port in fluid communication with at least one outlet port, wherein the inlet port is connectable to a fluid supply system external to the container unit and the outlet port connectable to a device for delivering persufflation fluid to an organ or body tissue when stored in the container unit. The device for delivering persufflation fluid to an organ or body tissue may comprise a cannula. The container unit may be provided with a venting port which is openable to reduce pressure within the storage region.

In an aspect, there is provided an automated method of persufflating an organ or body tissue portion, comprising the steps of: supplying a pressurised persufflation gas to an ex vivo organ or body tissue portion, isolated from a human or animal body, contained in a storage region of a container unit; determining if at least one parameter associated with the persufflation gas satisfies a respective predetermined condition; and automatically varying the parameter if the parameter does not satisfy the respective predetermined condition.

The step of determining if at least one parameter satisfies a respective predetermined condition may comprise comparing at least one of a measured flow rate parameter or measured pressure parameter of the supplied gas with a respective predetermined threshold value. The step of determining may comprise comparing a measured flow rate of the supplied gas with a respective predetermined flow rate threshold value and comparing a measure pressure of the supplied gas with a respective predetermined pressure threshold value. The respective predetermined threshold value may be associated with a respective minimum flow rate or minimum pressure of persufflating fluid required to persufflate the organ or body tissue portion. The minimum flow rate may be 10 mL/min.

The method may comprise supplying the persufflating gas automatically from a pressurised gas cylinder or concentrator member or pump member. The method may comprise providing the persufflating gas at a desired temperature prior to supplying the gas to the organ or body tissue portion. The method may comprise heating and/or cooling the persufflation gas prior to supplying the gas to the organ or body tissue portion. The method may comprise heating and/or cooling the persufflation gas via an active and/or passive cooling member. The method may comprise humidifying the persufflation gas prior to supplying the gas to the organ or body tissue portion. The method may comprise cooling and simultaneously dehumidifying the persufflation gas prior to supplying the gas to the organ or body tissue portion. The method may comprise the steps of: determining a temperature parameter associated with the storage region of a container unit; determining whether the temperature parameter satisfies a predetermined temperature condition; and activating an alert if the determined temperature parameter fails to satisfy the predetermined condition.

The method may comprise the steps of: determining whether the oxygen content of the persufflation gas satisfies a predetermined condition; and automatically varying the oxygen concentration if the oxygen content does not satisfy the predetermined condition. The method may comprise the step of activating an alert if it is determined that said at least one parameter associated with the persufflation gas does not satisfy the respective predetermined condition. The method may be a method of controlling anterograde persufflation of an organ or body tissue. The method may be a method of controlling retrograde persufflation of an organ or body tissue.

The step of supplying a pressurised persufflation gas to an ex vivo organ or body tissue portion, isolated from a human or animal body, contained in a storage region of a container unit may comprise supplying pressurised persufflation gas to a plurality of portions of the organ or body tissue. The steps of determining if at least one parameter associated with the persufflation gas satisfies a respective predetermined condition and automatically varying the parameter if the parameter does not satisfy the respective predetermined condition may be performed respectively and independently for each of the plurality of portions of the organ or body tissue. The method may be for anterograde persufflation of a pancreas for islet isolation.

In an aspect, there is provided a system for persufflating an organ or body tissue portion comprising: at least one source of pressurised persufflation gas for persufflating an ex vivo organ or body tissue portion, isolated from a human or animal body, contained in a storage region of a container unit; at least one regulator for regulating the supply of persufflating fluid to the organ or body tissue portion; at least one determiner for determining if at least one parameter associated with the persufflation gas satisfies a respective predetermined condition; at least one memory for storing at least one of said predetermined condition; and at least one controller configured to automatically vary the parameter if the parameter does not satisfy the respective predetermined condition by controlling the regulator.

The determiner may be configured to determine if at least one parameter satisfies a respective predetermined condition by comparing at least one of a measured flow rate parameter or a measured pressure parameter of the supplied gas with a respective predetermined threshold value. The respective predetermined threshold value may be associated with a respective minimum flow rate or minimum pressure of persufflating fluid required to persufflate the organ or body tissue portion. The system may be configured to supply a pressurised persufflation gas to an ex vivo organ or body tissue portion, isolated from a human or animal body, contained in a storage region of a container unit comprises supplying pressurised persufflation gas to a plurality of portions of an organ or body tissue. The controller may be configured to determine if at least one parameter associated with the persufflation gas satisfies a respective predetermined condition and to automatically vary the parameter if the parameter does not satisfy the respective predetermined condition respectively and independently for each of the plurality of portions of the organ or body tissue.

The system may be for anterograde persufflation of a pancreas for islet isolation. The source of pressurised persufflation gas may comprise a pressurised gas cylinder or concentrator member or pump member. The system may further comprise at least one heating element arranged to heat the persufflation gas prior to supplying the gas to the organ or body tissue portion and/or at least one cooling element arranged to cool the persufflation gas prior to supplying the gas to the organ or body tissue portion. The system may comprise a humidifying element arranged to humidify the persufflating gas prior to supplying the gas to the organ or body tissue portion. The system may comprise a temperature determiner arranged to determine a temperature parameter associated with the storage region of the container unit, wherein the controller is further configured to determine whether the temperature parameter satisfies a predetermined temperature condition. The system may further comprise an alert element, wherein the controller is configured to activate the alert element if the parameter does not satisfy the respective predetermined condition and/or the temperature parameter does not satisfy the predetermined temperature condition. The system may further comprise: an oxygen content determiner arranged to determine whether the oxygen content of the persufflation gas satisfies a predetermined condition; and wherein the controller is further configured to automatically vary the oxygen concentration if the oxygen content does not satisfy the predetermined condition.

Aspects of the present disclosure may provide one or more computer program products comprising instructions configured to program a processor to perform the steps of methods disclosed herein. Also disclosed herein are combinations of features of the above-described aspects. For example, features of the above-described container unit may also be disclosed in combination with the above-described features of the drape, the storage apparatus and/or the automated method. Likewise, features of the above-described drape may also be disclosed in combination with the above-described features of the container unit, the storage apparatus and/or the automated method. Likewise, features of the above-described storage apparatus may also be disclosed in combination with the above-described features of the drape, the container unit and/or the automated method. Likewise, features of the above-described automated method may also be disclosed in combination with the above-described features of the drape, the storage apparatus and/or the container unit.

### Figures

Some examples of the present disclosure will now be described, by way of example only, with reference to the figures, in which:

FIG. 1 shows a portable apparatus for storing an organ or body tissue in a closed configuration;

FIG. 2 is a sectional view of the portable apparatus shown in FIG. 1;

FIG. 3 is a sectional view of a portion of the portable apparatus shown in FIGS. 1 and 2 in an open configuration;

FIG. 4 is a schematic representation of a layout of a portable apparatus;

FIG. 5 is a plan view of a portion of a container unit;

FIG. 6 is sectional view along A-A of FIG. 5;

FIG. 7 is a sectional view along B-B of FIG. 5;

FIG. 8A shows a plan view of a fluid processing device which also illustrates the location of some hidden features;

FIG. 8B is a sectional view along B-B of FIG. 8A;

FIG. 8C is an end view of the fluid processing device shown in FIG. 8A;

FIG. 8D is an end view of the fluid processing device shown in FIG. 8A;

FIG. 8E is a side view of the fluid processing device shown in FIG. 8A;

FIG. 8F is a sectional view of the fluid processing device shown in FIG. 8A from the underside;

FIG. 9 shows an alternative example of a fluid processing device;

FIG. 10 shows a base unit in which the base unit is in an open configuration with a lid removed;

FIG. 11 shows a surgical drape;

FIG. 12A is a schematic representation showing a partial sectional view of a portion of a base unit and a container unit in a first configuration;

FIG. 12B corresponds to FIG. 12A and shows the base unit and the container unit in a second configuration;

FIG. 12C corresponds to FIG. 12A and shows the base unit and the container unit in a third configuration;

FIG. 12D corresponds to FIG. 12A and shows the base unit and the container unit in a fourth configuration;

FIG. 12E corresponds to FIG. 12A and shows the base unit and the container unit in a fifth configuration;

FIG. 12F corresponds to FIG. 12A and shows the base unit and the container unit in a sixth configuration;

FIG. 13A corresponds to FIG. 12A and shows the base unit and the container unit in an seventh configuration;

FIG. 13B corresponds to FIG. 12A and shows the base unit and the container unit in a eighth configuration;

FIGS. 14A shows a cooperation between a base unit and a first container unit;

FIG. 14B shows cooperation between a base unit and a second container unit;

FIGS. 15A to 15E (numbered from left to right and top to bottom) show stages of a surgical drape being unfolded;

FIG. 16 is a schematic representation of a portion of the apparatus shown in FIGS. 1 and 2 during use;

FIG. 17 shows a user interface;

FIG. 18 shows components of a system for persufflating an organ or body tissue;

FIG. 19 illustrates a method of persufflating an organ or body tissue;

FIG. 20 is a schematic illustration of a portion of an alternative example of a container unit;

FIG. 21 is a projection view of a portion of a flow passageway shown in FIG. 20;

FIG. 22 is a schematic representation of a portion of an example of a container unit;

FIG. 23A is a schematic representation of a layout of certain components of a container unit;

FIG. 23B is a schematic representation of a layout of certain components of a container unit;

FIG. 23C is a schematic representation of a layout of certain components of a container unit;

FIG. 24 is a schematic representation of a portion of a container unit; and

FIG. 25 is a schematic representation of a portion of a system comprising certain components of a container unit.

In the drawings like reference numerals are used to indicate like elements.

### SPECIFIC DESCRIPTION

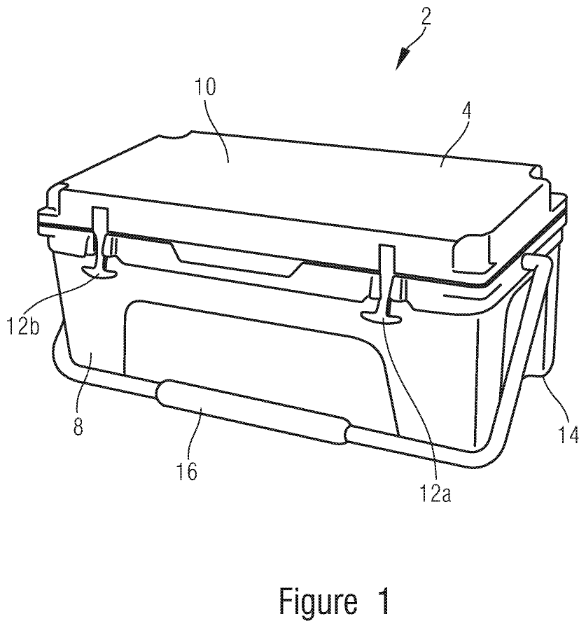

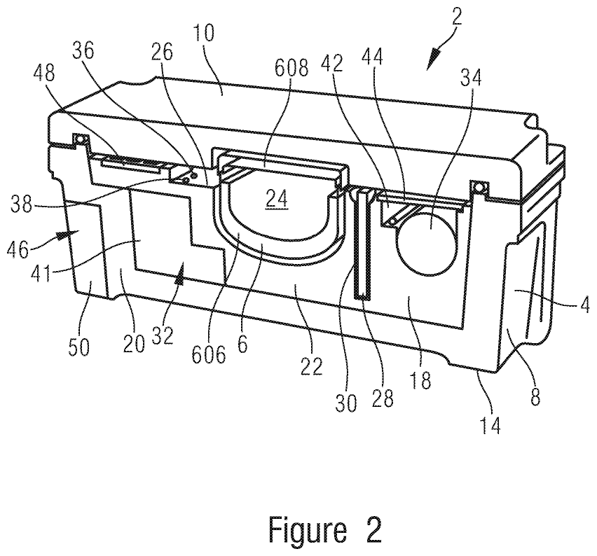

FIGS. 1 and 2 show a portable storage apparatus 2 for storing an organ or body tissue of a human or animal. The apparatus 2 comprises a base unit 4 and a container unit 6 (visible in FIG. 2). The base unit 4 (which may also be referred to as a retained unit) comprises a lower portion 8 and a detachable base unit lid member 10 having clasps 12a, 12b which secure the lid to the lower portion. The base unit 4 is generally oblong and has a generally flat lower surface 14 on which the base unit 4 is supported. The base unit 4 also has a carrying handle 16 which is connected at opposite ends of the lower portion 8.

The base unit 4 comprises an inner portion 18 and an outer portion 20. The inner portion 18 comprises a docking portion 22 which is configured to receive the container unit 6. The docking portion 22 defines a storage region in the form of a storage cavity 24 having an opening 26 at the top of the storage cavity 24 through which the container unit 6 can be inserted into the storage cavity 24.

Cooling elements 28 in the form of ice-packs are arranged adjacent the docking portion 22 in order to passively cool the container unit 6 once it is received within the storage cavity 24. The cooling elements 28 are housed within respective vertical slots 30 provided in the inner portion 18 such that the cooling elements 28 can be inserted and removed from the slots 30, as required.

The base unit 4 further comprises a fluid supply system 32. The fluid supply system 32 comprises a fluid source 34 connected to a first outlet port 36, a second outlet port 38, a third outlet port 40 and at least one regulator and associated conduits (indicated generally by reference 41 and described later in further detail). The fluid supply system 32 may optionally include a fluid processor such as a humidifier and/or a cooling/heating element. The cooling elements 28 (and heating elements if provided) may be arranged to cool the persufflating gas as it passes through the fluid supply system 32 prior to reaching the outlet ports 36, 38, 40. The fluid source 34 comprises a gas canister which is located within a fluid source storage cavity 42 defined by the inner portion 18. The fluid source storage cavity 42 is provided with a removable transparent cover 44.

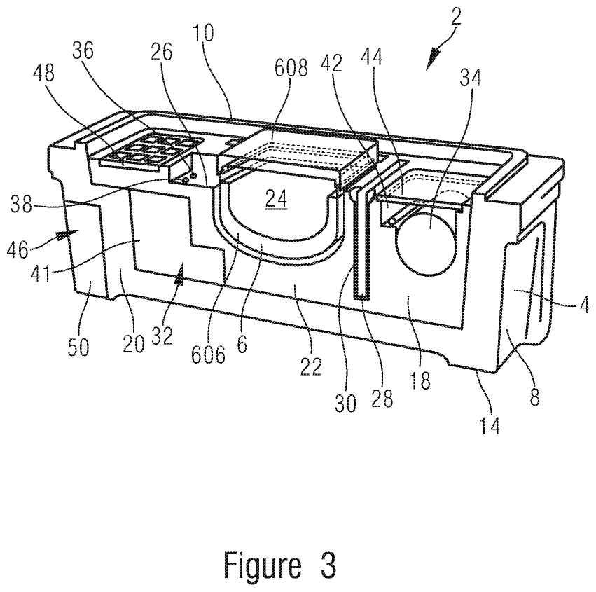

FIG. 3 shows the base unit 4 with the lid member 10 removed so that the top of the lower portion 8 is visible. The base unit 4 further comprises a control system 46 which comprise a user interface 48 in the form of key pad, a controller and power source (indicated generally by reference 50). The control system is configured to monitor and control portable apparatus 2. The heat generating components of the control system, such as the power source, are located externally of the outer portion 20.

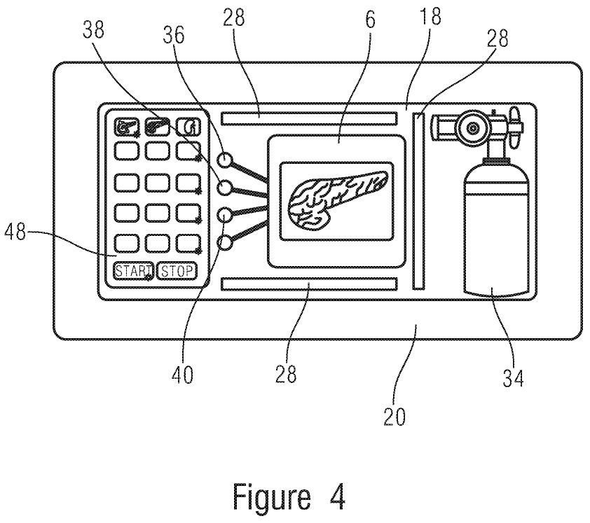

FIG. 4 shows an example layout of the base unit 4 in which like reference figures are used to refer to like parts.

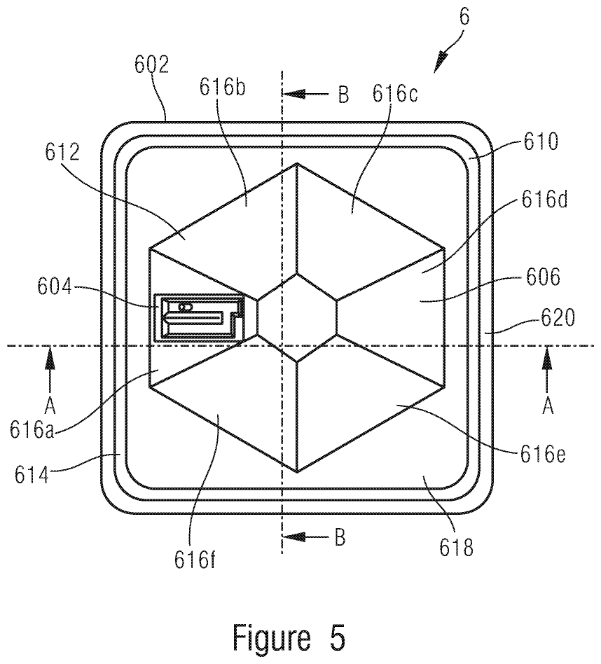

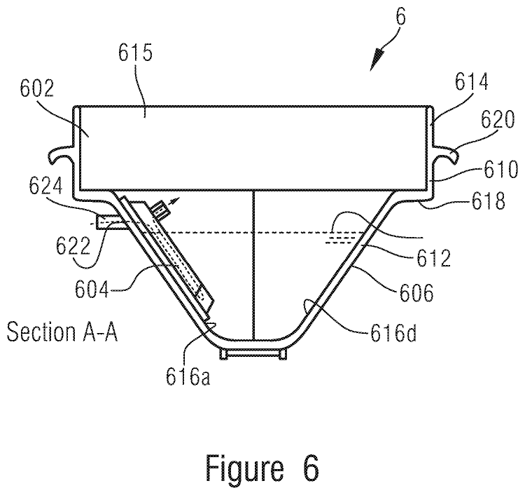

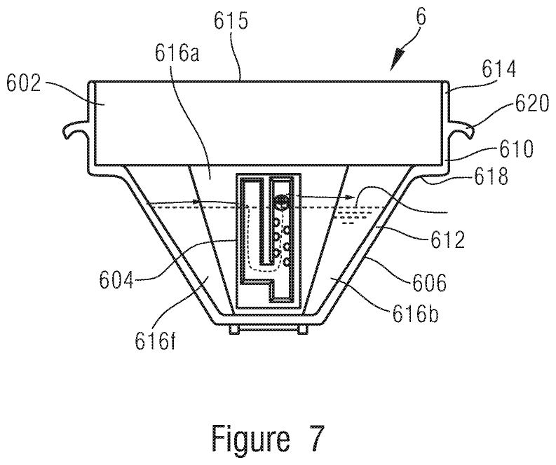

FIGS. 5, 6 and 7 show the container unit 6 in more detail. The container unit 6 (which may also be referred to as a disposable unit or a consumable unit or a primary organ container) comprises a container body 602 and a fluid processing device 604. The container body 602 comprises a storing portion 606 and a container unit lid member 608 (not shown in FIG. 5, 6 or 7, but shown as part of a general arrangement in FIGS. 2 and 3).

The storing portion 606 has an outer wall 610 which forms a frusto-conical lower portion 612 and an upper portion 614 and defines an opening 615 through which an organ can be inserted or removed from the storing portion 606. It will be appreciated that other shapes for the storing unit could be used such as hemispherical or square or ellipsoidal or the like.

The lower portion 612 forms a bowl which defines a cavity in which the organ or body tissue is stored. The lower portion 612 has a hexagonal cross-section (when viewed from the top) and defines six substantially flat inner surfaces 616a, 616b, 616c, 616d, 616e, 616f.

The upper portion 614 has a substantially rectangular cross-section (when viewed from the top). A connection portion 611 of the outer wall 610 forming the upper portion 614 extends horizontally to form a ledge 618. The upper portion 614 also comprises a retaining lip 620 to which the container unit lid member 608 is secured.

A container inlet port 622 is provided through the outer wall of the lower portion 612. The inlet port 622 is located immediately below the ledge 618 and comprises a connector 624 which extends outwardly from the side of the portion of the outer wall 610 forming the lower portion 612. It will be appreciated that the connector can extend further or less distance in length than that shown and can terminate in a connection element such as a male or female part of a quick disconnect connector.

The storing portion 606 is formed from a single piece of material, such as a polypropylene or other relatively inexpensive plastic. Similarly, the container unit lid member 608 is also formed from a single piece of material, such as a polypropylene or other relatively inexpensive plastic. In the present example the storing portion 606 and the container unit lid member 608 are each formed by a moulding process.

The fluid processing device 604 is fixed to one of the inner surfaces 616a of the lower portion 612, for example by an adhesive, ultrasonic weld or other suitable means.

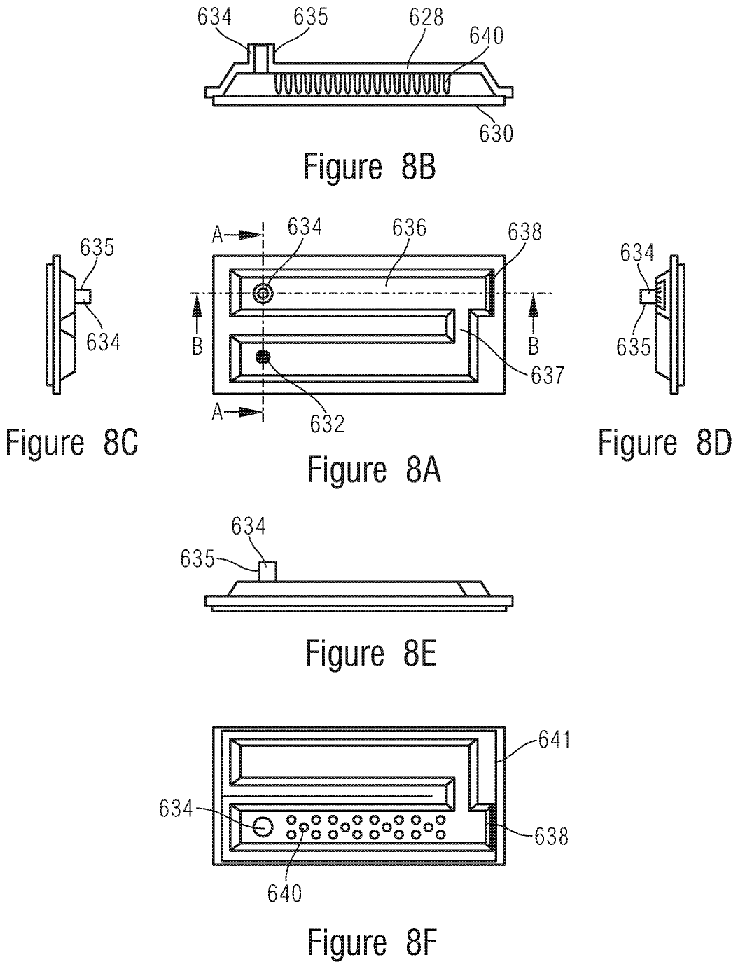

The fluid processing device 604 is shown in isolation in FIGS. 8A to 8F.

The fluid processing device 604 comprises an upper wall 628 and a bead 630, as shown in FIG. 8B. The bead 630 is secured via a fluid tight seal to a portion of the inner surface of the wall of the lower portion 612 of the wall of the storage portion. Alternatively the fluid processing device can have a lower base plate. A first inlet port 632 is provided though the lower wall and an outlet port 634 is provided through the upper wall 628. The outlet port 634 comprises a connector 635 for connecting to a delivery device such as a delivery tube and cannula arrangement. The first inlet port 632 and the outlet port 634 are provided in fluid communication with each other by an internal passageway 636 defined between the upper wall 628 and the lower wall 630.

The internal passageway 636 has a serpentine arrangement, meaning that the internal passageway 636 comprises at least one bend through which the internal passageway 636 turns through at least 90 degrees. In the example shown, the passageway 636 turns through a bend 637 of 180 degrees. The fluid processing device 604 is elongate and the first inlet port 632 and the outlet port 634 are provided at the same end of the of the fluid processing device 604 and the internal passageway 636 extends from the first inlet port 632 along the fluid processing device 604 then turns through 180 degrees and extends back along the fluid processing device 604 to the outlet port 634. It will be appreciated that as an alternative the internal passageway may have a linear arrangement.

A second inlet port 638 is provided at the end of the fluid processing device 604 which is opposite the end at which the first inlet port 632 and the outlet port 634 are provided. The second inlet port 638 is an elongate slot (termed a “letterbox”) which is arranged to allow fluid to enter the internal passageway 636 in the region of the turn 637 of the internal passageway 636. Other shapes of port can be utilised.

Baffles 640 are provided in the region of the internal passageway 636 which extends from the bend 637 to the outlet port 634. Each baffle 640 comprises a cylindrical pin (but may be a conical frustra) which extends from the upper wall 638 towards the lower wall 630. In the example shown, the baffles 640 are formed integrally with the upper wall 638 and each baffle 640 extends across substantially all of the internal passageway 636 in a direction which is perpendicular to the upper wall 638. It will be appreciated that the baffles 640 may be formed integrally with the upper wall 638 and/or the lower wall 630 and may extend partially across the internal passageway 636. The internal passageway 636 (regardless of whether it is straight or bent once or u-shaped or serpentine) thus includes one or more elements that break up any bubbles provided by the incoming gas. The bubble break up elements can be pegs or beads or plates or hemispherical or tapered projections or other such elements that operate to create a longer pathway in the internal passageway and/or provide shattering surfaces and/or define interspatial gaps that preclude transfer of large bubbles.

As the bowl-like storage region in the container unit fills with a preservation liquid the liquid automatically fills the space defined by the internal passageway. The passageway and fluid processing device in general thus forms an integral part of the disposable container unit.

The upper wall 628 and a lower wall 630 may be joined at their peripheries by a sealing bead 641, such as a bead formed by ultrasonic welding of the upper wall 628 and a lower wall 630 together as shown in FIG. 8F.

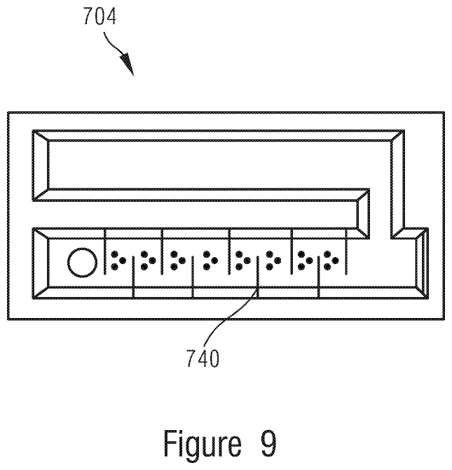

FIG. 9 shows an alternative example of a fluid processing device 704 in which baffles 740 are arranged to define a zig-zag flow path along a flow passageway.

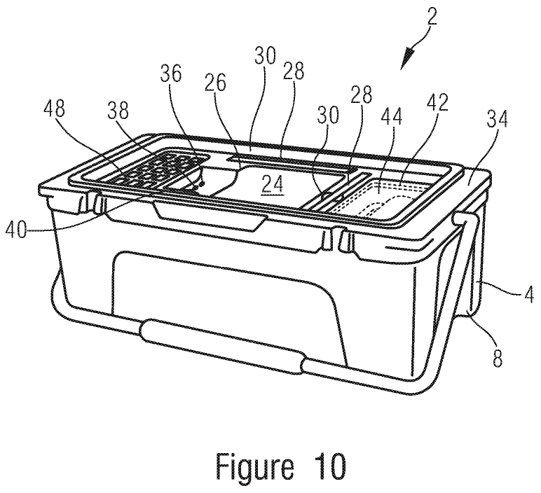

Prior to use, the base unit lid member 10 is removed from the lower portion 8 of the base unit 4, as shown in FIG. 10. The container unit 6 is then inserted into the storage cavity 24, as shown in FIG. 3 so that the container unit 6 is held securely within the docking portion 22. The container unit lid member 608 (not shown in FIG. 3) comes packaged separately.

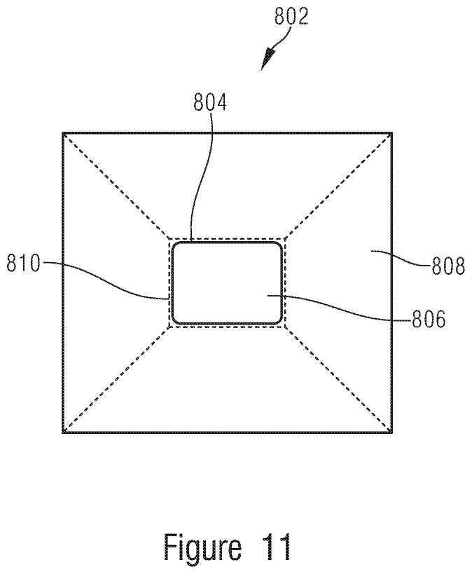

A surgical drape 802, as shown in FIG. 11, is pre-packaged as a unit already secured to the container unit 6 around a peripheral edge region at a centre of the drape.

A ‘surgical drape’ in the context of the present disclosure is a flexible drape (typically made of cloth) that can be made sterile, or treated so that it is sterile, for use in a surgical environment to provide a sterile surface in the area of a surgical procedure and to provide a barrier against migration of bacteria.

In more detail the surgical drape 802 has a central securing portion 804 through which is provided an aperture 806, and peripheral outer portion 808 which surrounds the securing portion 804. A frangible portion 810 which comprises a perforated line extends circumferentially about the securing portion 804 thereby providing a relatively weak portion of the drape 802 between the securing portion 804 and the outer portion 808. The securing portion 804 is provided with an adhesive on an upper surface of the securing portion 804 about the periphery of the aperture 806.

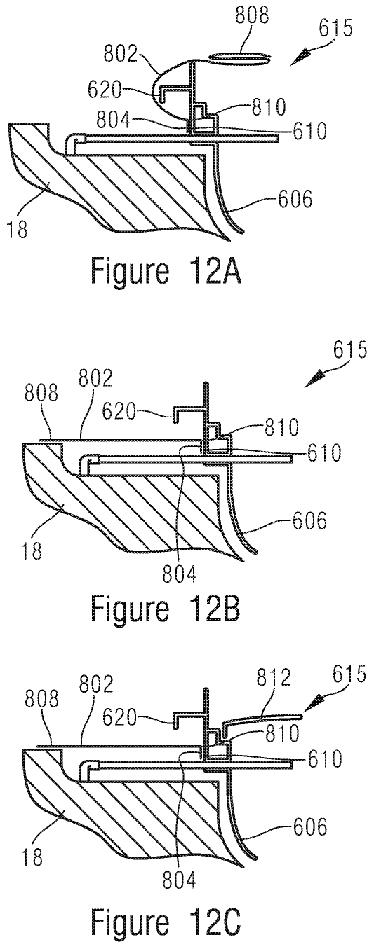

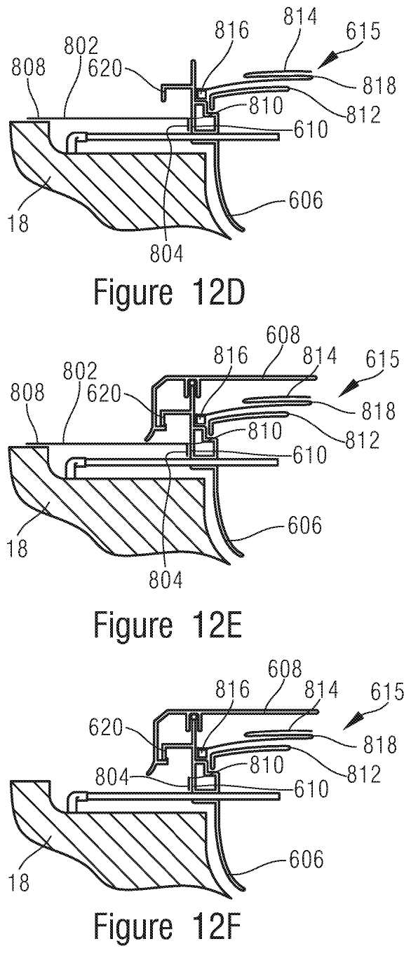

The securing portion 804 is secured to an outer surface of the outer wall 610 of the storing portion 606 of the container body 602 below the retaining lip 620, as shown in FIG. 12A (note that only a portion of the surgical drape is shown attached to the outer wall on one side of the container unit and that the pipe extending through the outer wall 610 is a representation of how the fluid supply system my supply persufflating fluid to the inside of the container unit 6 an is not an accurate representation of the example shown in the previous Figures), so that the securing portion 804 surrounds the opening 615 of the storing portion 606.

Once secured, the surgical drape 802 is unfolded so that the outer portion 808 overlies the base unit 4 and so provides a sterile surface in the vicinity of the container unit 6 ensuring sterility as shown in FIG. 12B. In this way a disposable container unit can be located in the non-consumable unit and the attached drape opened out away from the open mouth of the container unit provided under the lid. The drape can be spread out by a surgeon prior to location of an organ or tissue portion in the central space of the container unit. In its opened out or spread state the sterile surgical drape covers and thus protects the whole of the upper surface of the non-consumable base unit 4. The drape thus protects the user interface 48 and top and sides of the base unit. A surgeon can then duly locate a selected one body organ or body tissue position in the container unit having first duly selected a container unit having the requisite dimensions and/or shape for the organ or tissue portion concerned. For example a container unit for a liver will have a larger internal space than a container unit for a kidney.

Prior to cross-clamp, initial set-up of the system on a ‘back-bench’ will be performed by suitable organ retrieval support personnel.

Following cross-clamp and flushing in accordance with local protocols, the organ/tissue will be removed to the back-bench for trimming. If en bloc the organ/tissue will be dissected from any other organs/tissues removed with it and then moved to the apparatus 2. Gross dissection trimming may occur here according to surgeon preference. If not en bloc then the organ/tissue will be directly removed into the apparatus 2.

The apparatus 2 will be powered up and the organ/tissue cannulated according to defined protocols. This may be performed with any cannula according to physician preferences but will involve an airtight seal being formed between the device outlet and the inlet into the tissue; typically a significant artery or vein. Upon attachment, the system will run according to the desired automation algorithm and the surgeon will identify any significant leaks and ligate. Ligation may be according to any locally accepted practices including but not limited to tieing with a suture, stitching with a suture, vascular/tissue sealant, other means such as ligaclip/staple.

If indicated, the surgeon will ensure that no inlet is occluded by any means including but not limited to: twist in the vessel, tip occlusion due to being pressed into the vascular wall/tissue, pinching/clamping of a tube, etc.

Any amount of desired dissection/trimming of the organ/tissue in preparation for transplantation/cell isolation may be performed during system function including either upon connection or following receipt. Once the organ indicates no occlusions and has no obvious vascular leaks (indicated by rapid bubbling typically on or around a major vessel/cannulation site) and exhibits the desired outflow; the organ should be packaged for maintenance and transport within the apparatus 2.

In an alternative example, the drape 802 may be pre-secured to the container unit 6 so that it can be packaged together with the container unit 6 as a single-use sterile article.

An organ or body tissue, such as an organ or body tissue that has been extracted from a body, is surgically prepared, for example excess tissue trimmed away, and placed through the opening 615 into the storing portion 606.

The storing portion 606 is filled with a preserving fluid either before or after the organ/body tissue has been placed into the storing portion 606. An organ will typically float in the preservation fluid PF, but may be constrained by a mesh, or similar, around it avoiding direct contact with the fluid.

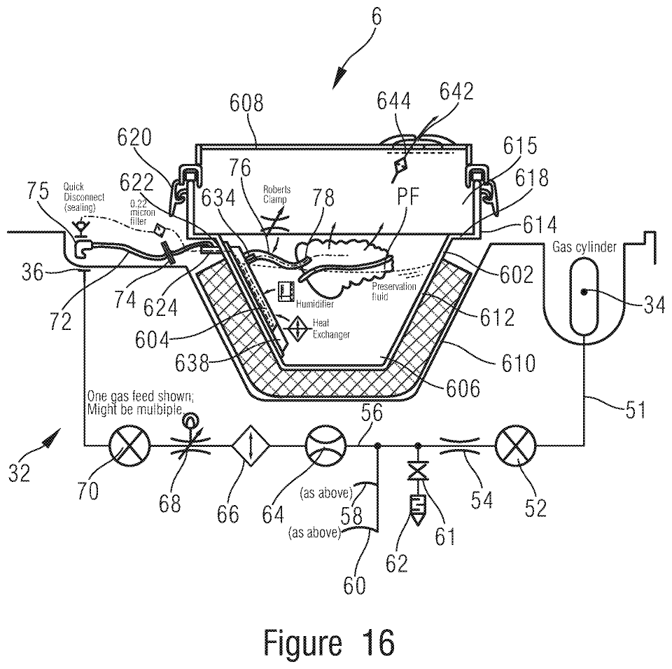

Respective cannulas are then connected to the outlet port 634 of the fluid processing device 604, as described later with reference to FIG. 16, and an internal cover 812 is placed within the upper portion 614 such that it is supported by the ledge 618, as shown in FIG. 12D, so as to enclose the organ/body tissue within the storing portion 606.

A second surgical drape 814 having a construction which is similar to the surgical drape 802 is provided in a folded configuration over the internal cover 812. The second surgical drape 814 has a central securing portion 816 having an adhesive on its underside and a peripheral outer portion 818. In the example shown, the second surgical drape does not have a frangible portion separating the two. The securing portion 816 is secured to an upper surface of the ledge 618, which is shown as a peripheral stepped portion of ledge in FIGS. 12A to 12F. Once secured, the container unit lid member 608 is secured to the retaining lip 620, thereby sealing the organ/body tissue and within the storing portion 606, as shown in FIG. 12E.

The peripheral outer portion 808 of the drape 802 is then torn away from the securing portion 804 along the frangible portion 810, as shown in FIG. 12F. The base unit 4 is then ready to be operated in order to start persufflation of the organ/body tissue.

FIG. 16 is a schematic representation of a portion of the apparatus 2 once assembled with an organ placed within the container unit 6.

FIG. 16 shows components of the fluid supply system 32 in more detail. The fluid supply system 32 comprises the fluid source 34 which is connected in series by a primary flow line 51 to a primary pressure meter 52, a primary adjustable regulator 54 in the form of a pinch valve, and first, second and third fluid supply lines 56, 58, 60 which are connected respectively to the first outlet port 36, second outlet port 38 and third outlet port 40. A vent 61 and a silencer 62 are provided between the primary adjustable regulator 54 and a junction with the first, second and third fluid supply lines 56, 58, 60. The first, second and third fluid supply lines 56, 58, 60 are arranged in parallel.

The first fluid supply line 56 comprises a flow meter 64, a heat exchanger 66, a secondary adjustable regulator 68 in the form of a pinch valve, and a secondary pressure meter 70. The second and third fluid supply lines 58, 60 are identical to the first fluid supply line 56.

A first flexible tube 72 connects the first outlet port 36 to the connector 624 of the container unit 6. The flexible tube is fitted with a filter 74 and a ‘quick disconnect’ connector 75. The filter is a 0.22 micron filter.

A second flexible tube 76 connects the outlet port 634 of the fluid processing device 604 to a cannula 78. It will be appreciated that as an alternative the second flexible tube 76 and cannula 78 could be provided as a single element. A flow regulator, in the form of a Roberts clamp, may fitted to the second flexible tube 76. The cannula 78 is inserted into a blood vessel of an organ or body tissue stored within the storing portion 606. The second and third fluid supply lines 58, 60 are not in use.

The container lid member 608 comprises a vent 642 which is fitted with a filter 644. The filter may be a 0.22 micron filter.

A preservation fluid PF (which may be an aqueous media) is provided within the storing portion 606. The preservation fluid may be added either before or after the organ/body tissue has been placed within the storing portion 606. In FIG. 16 the storing portion 606 is filled with preservation fluid to a level which is above the second inlet port 638 and below the outlet port 634 of the fluid processing device 604. The preservation fluid PF therefore fills the internal passageway 636 of the fluid processing device 604 up to the level of the preservation fluid PF within the storing portion 606. The storing portion 606 may be prefilled with persufflation fluid PF so that it arrives sealed, sterile and ready for immediate use. The fluid processing device 604 therefore acts as a gas humidifier and heat exchanger to cool or heat and humidify persufflation gas within the fluid processing device 604 using the preservation fluid PF. It will be appreciated that as an alternative the level of the preservation fluid could be above the inlet and/or outlet port. The anatomy of the organ or tissue portion associated for use with any one container unit can determine the location of outlet port and thus a site for projection of an outwardly extending lumen designed to provide a respective gas supply for an inlet to the organ or tissue portion.

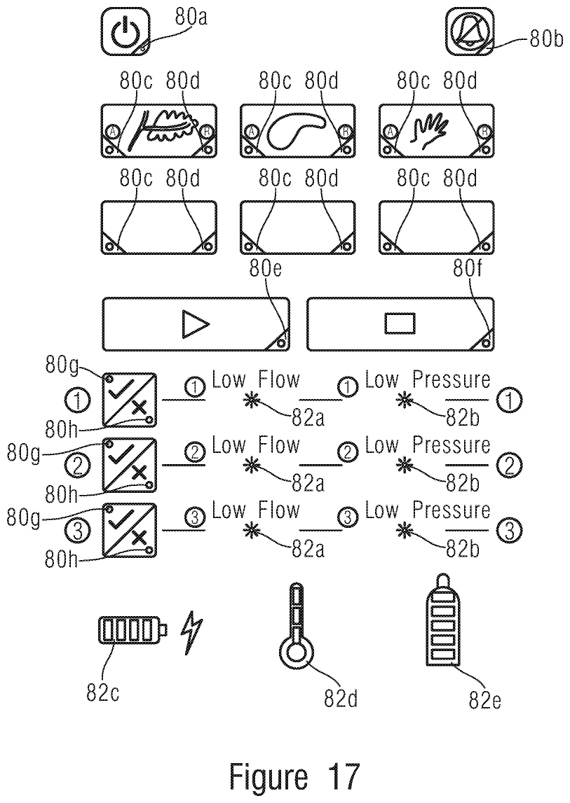

FIG. 17 illustrates an example of a key pad layout for the user interface 48. The user interface 48 comprises a plurality of buttons 80a to 80h and a plurality of graphical indicators 82a-82e. Each button 80a to 80h is a membrane button having an integral LED configured to illuminate the button 80a to 80h when actuated.

The user interface 48 comprises a standby button 80a, an alarm silencing button 80b, a plurality of organ/body tissue selection buttons 80c, 80d, an activation (‘start’) button 80e, a deactivation (‘end’) button 80f, and a plurality of line activation and deactivation buttons 80g, 80h. Alternatively the user interface (VI) can be wholly or partly provided by a touchscreen which may or may not have physical buttons on the side. It will be appreciated in the context of the present disclosure that a mobile telecommunication handset (such as a mobile phone or tablet) may be connected (e.g. via a communication interface such as a network, or via Bluetooth (RTM)). The mobile telecommunication handset may provide a user interface for the apparatus.

The standby button 80a is configured to power the control system 46 on and off. The alarm silencing button 80b is configured to deactivate or activate one or more selected alerts such as alarms. Organ/body tissue selection buttons 80c, 80d are provided for each type of organ/body tissue (for example, a pancreas, kidney or a limb) for which the apparatus 2 is to be used. In the example shown, there are first and second organ/body tissue buttons 80c, 80d are provided for each type of organ/body tissue. The first organ/body tissue button 80c is selectable for providing anterograde persufflation of the organ/body tissue and the second organ/body tissue button 80d is selectable for providing retrograde persufflation of the organ/body tissue. Blank organ/body tissue buttons 80c, 80d may be provided for future updates to the control system 46 to enable persufflation of organs having different persufflation parameters.

The activation button 80e is configured to commence flow of a persufflation fluid to the organ/body tissue stored in the apparatus 2. The deactivation button is configured to arrest flow of a persufflation fluid to the organ/body tissue stored in the apparatus 2.

A line activation button 80g and a line deactivation button 80h may be provided for activating/deactivating each fluid supply line 56, 58, 60 connected to the organ/body tissue. The user interface 48 comprises a ‘low flow’ indicator 82a and a ‘low pressure’ indicator 82b for each fluid supply line 56, 58, 60. The user interface 48 also comprises a power level indicator 82c, a temperature indicator 82d for indicating the internal temperature within the space 22 and a pressure indicator 82e for indicating the pressure and thus remaining contents of the fluid source. Activation of these indicators may encompass a time element so as not to trigger on a single aberrant reading but for example to either trigger upon a given threshold for any of these values or to trigger if an average deviation of >30% is observed over any 3 minute period.

The base unit 4 thus provides a thermally protected area 22. Heating and/or cooling elements which may be active or passive help modulate temperature in the thermally protected area. For example, a Peltier device may be used as an active heating/cooling device. Aptly the temperature can be at least slightly heated or slightly cooled relative to ambient temperature. In this way if during the day in a hot climate temperatures in the air surrounding the base unit rise the temperature in the zone within the base unit close to the disposable container unit can be kept within an acceptable temperature range.

Aptly one or more temperature sensors are located in the climate controlled zone to provide temperature feedback to the controller. If only passive cooling or heating is used a temperature sensing feedback control can be omitted.

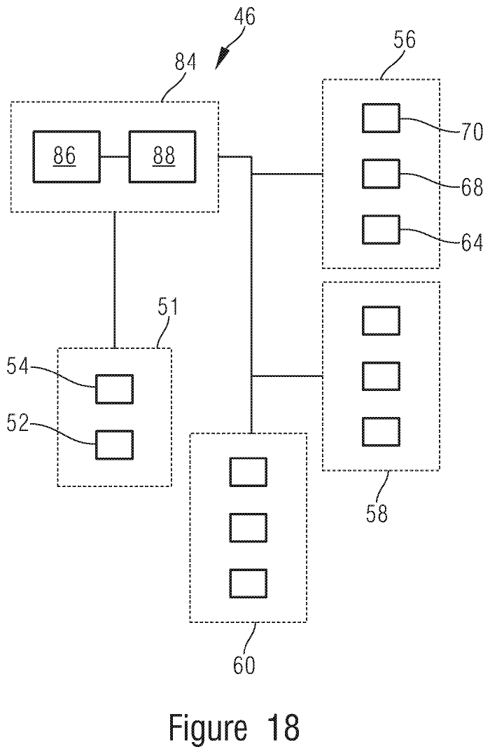

A schematic representation of the control system 46 is shown in FIG. 18. The control system 46 comprises a control module 84 which includes a controller 86 and a memory 88. The controller 86 is configured to determine pressure of the fluid source 34 based on an output from the primary pressure meter 52, and to control the primary adjustable regulator 54 in accordance with the determined pressure and a predetermined pressure stored by the memory 88.

The controller 86 is further configured to determine pressure of persufflating fluid within the first fluid supply line 56 based on an output from the secondary pressure meter 70, and to control the secondary adjustable regulator 68 in accordance with said determined pressure and a further predetermined pressure stored by the memory 88. Aptly the controller can be likewise utilised to determine and control pressure and/or humidity. These can be controlled independently

The controller 86 is further configured to determine flow rate of persufflating fluid within the first fluid supply line 56 based on an output from the flow meter 64 and to control the secondary adjustable regulator 68 in accordance with said determined flow rate and a predetermined flow rate.

The controller 86 is also configured to monitor and control the flow regulating components of the second and third supply lines 58, 60 in the same manner as the first supply line 56.

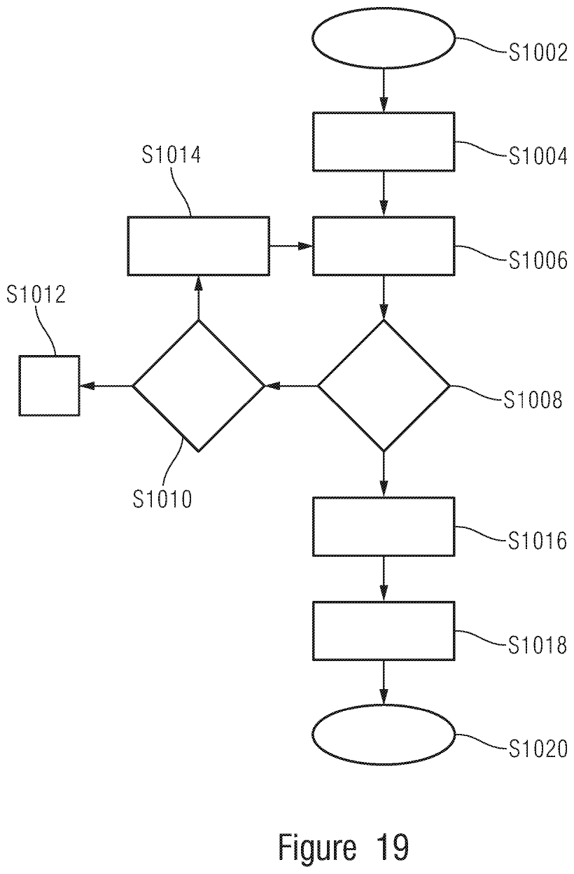

Once the organ has been connected to the fluid supply system 32 as described with reference to FIG. 16, persufflation of the organ/body tissue is conducted in accordance with the method shown in FIG. 19.

In step S1002, the standby button 80a is actuated in order to supply power from the power source to the remainder of the control system 46. In this step, the alarm silencing button 80b may be pressed to select whether pre-set alarms are active or inactive according to preference.

In step S1004, the appropriate organ/body tissue selection button is actuated in order to select the correct perfusion control function dependent on the type of organ to be persufflated and the type of persufflation required (i.e. anterograde or retrograde persufflation). The controller 86 is preprogramed to selectively supply persufflation fluid to the first, second and/or third supply lines 56, 58, 60 at a predetermined pressure and/or flow rate. Aptly the range of flow rate that can be provided and a desired flow rate set is 10 to 100 ml/min. The larger flow rate may for example be useful for a liver which is a relatively big aerobically active organ. Aptly the range is 15 to 30 ml/min.

It is possible as an alternative to just determine a single parameter associated with persufflation. For example for retrograde persufflation techniques/operation only pressure is controlled and flow rate is not controlled. Alternatively for anterograde persufflation flow rate alone is determined and pressure is left uncontrolled.

For example, if anterograde persufflation of a pancreas is selected, the controller may be configured to supply fluid to all three supply lines 56, 58, 60 by controlling the secondary adjustable regulator 68 of each supply line 56, 58, 60 to supply a persufflation fluid at stepped increases in pressure between 25 mmHg and 60 mmHg until a predetermined flow rate, for example 25 mL/min, is reached for each supply line.

If anterograde persufflation of a kidney is selected, the controller 86 may be configured to supply fluid to two of the three supply lines 56, 58, 60 in order to supply persufflating fluid to a primary renal artery and an accessory vessel by controlling the secondary adjustable regulator 68 of each supply line 56, 58, 60 to supply a persufflation fluid at stepped increases in pressure between 60 mmHg and 80 mmHg until a predetermined flow rate, for example 25 mL/min, is reached for each supply line.

If anterograde persufflation of a heart is selected, the controller 86 may be configured to supply fluid to supply lines 56, 58, 60 connected to each coronary artery by controlling the secondary adjustable regulator 68 of each supply line 56, 58, 60 to supply a persufflation fluid at stepped increases in pressure between 64 mmHg and 85 mmHg until a predetermined flow rate, for example 25 mL/min, is reached for each supply line.

If retrograde persufflation of a liver is selected, the controller may be configured to supply fluid to the desired number of supply lines 56, 58, 60 by controlling the secondary adjustable regulator 68 of each supply line 56, 58, 60 to supply a persufflation fluid at a set pressure of 18 mmHg.

If retrograde persufflation of a kidney is selected, the controller may be configured to supply fluid to the desired number of supply lines 56, 58, 60 by controlling the secondary adjustable regulator 68 of each supply line 56, 58, 60 to supply a persufflation fluid at a set pressure of 18 mmHg.

It is envisaged that each of the supply lines 56, 58, 60 may be controlled independently in order to supply a persufflation fluid having a predetermined pressure and/or flow rate to a particular part of an organ or body tissue to which each respective supply line 56, 58, 60 is connected.

In each of the above examples, the flow rate and pressure is determined at step S1006. It is then determined in step S1008 whether the flow rate and pressure satisfy the predetermined flow rate and the predetermined pressure. If the determined flow rate and/or the determined pressure does not satisfy the predetermined flow rate, it is then determined in step S1010 whether the determined flow rate and/or the determined pressure satisfy an alert condition. If it is determined in step S1010 that an alert condition is met, an alert is activated in step S1012 to trigger inspection for a leak or obstruction. The alert may be a visual or audible alert. If an alert condition is not met, the pressure and/or flow rate is incremented to a predetermined intermediate value or by a predetermined amount in step S1014. The necessary steps S1006 through S1014 are then repeated until the flow rate satisfies the predetermined flow rate. Aptly the pressure (or indeed one or more other parameters) is increased and/or decreased continually as needed throughout persufflation. If it is determined in step S1008 that the flow rate and pressure satisfy the predetermined flow rate and the predetermined pressure, operation proceeds to step S1016 in which the system enters a maintenance mode to supply the persufflation fluid at the predetermined flow rate and pressure.

In each instance, the temperature within the cavity within which the organ/body tissue is stored is monitored and indicated to a user via the temperature indicator 82d. If it is determined that the temperature is outside of a desired temperature ranges, such as between 4 degrees centigrade and 20 degrees centigrade, for example between 4 degrees centigrade and 8 degrees centigrade, the controller 86 activates an alert. In alternative examples comprising an active heating element and/or an active cooling element, the heating element/cooling element may be controlled to maintain the temperature of the organ/body tissue within the desired temperature range.

A persufflation fluid passes through the internal passageway 636 of the fluid processing device 604 it travels through the convoluted path provided by the baffles 640 which cause the persufflating fluid to be both cooled and humidified by the preservation fluid PF within the internal passageway 636. Preferably, the gas is humidified to saturation.

It is anticipated that that the persufflation fluid will comprises an oxygen gas mixture comprising 40% oxygen. This provides a satisfactory balance between providing satisfactory oxygenation of tissue without being toxic. Aptly the oxygen content is between 20% to 95% oxygen generally. If anterograde operation is being used the oxygen range is 30% to 40%. If retrograde operation is being used 90% to 95% oxygen is used.

Once the persufflating fluid is being supplied to the organ/body tissue at the desired flow rate and at the desired pressure, the base unit lid member 10 is then secured to the lower portion 8 ready for transport.

The control system 46 remains active throughout transportation to monitor divergences in the flow rate and pressure and control the flow rate and/or pressure in response and also to alert an operator when the pressure, temperature or flow rate of the persufflating fluid is abnormal.

Subsequent to a surgeon duly locating an organ or body tissue portion in the container and sealing it with the lid of the container unit the base unit and container unit system constantly governs itself. During this period the system constantly and automatically tries to maintain one or more parameters on each line supplying persufflation gas to a respective region of an organ or body tissue at a desired target value or within a desired range bounding a predetermined target threshold value.

The controller optionally includes a logger which stores values for monitored parameters. Aptly a time/date entry is stored along with recorded parameter (such as pressure, temperature, flow rate and/or humidity) values in step S1018. Alarm codes associated with leak or blockage alarms can also be stored. This provides helpful data for a surgeon subsequent to transport of the organ or tissue portion is likely to still be fit for purpose.

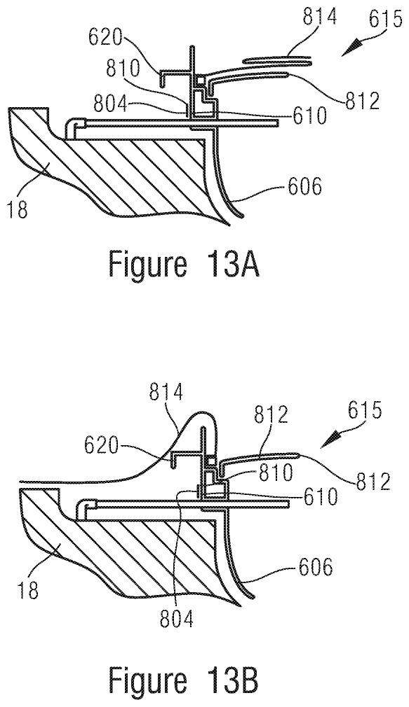

On arrival at an intended destination or after the organ/body tissue has been held in storage for a desired period of time, the base unit lid member 10 is removed. The container unit lid member 608 is also removed to reveal the second surgical drape 814, as shown in FIG. 13A. The second surgical drape 814 is then unfolded so that it lies across the base unit 4 and so provides a sterile work surface, as shown in FIG. 13B. The internal cover 812 is then removed and the organ/body tissue is removed from the storing portion 606 for inspection and further processing.

For example, while the system is running, the surgeon may wish to perform further dissection of the organ/body tissue in preparation for implantation/cell isolation. The organ/body tissue may then be removed from the cannula and the system shut down. This may be done according to local policy. At this point, it may be desirable to manually flush the organ/tissue with liquid/preservation solution to purge the gas from it. This may be done according to surgeon preference with a syringe, by hanging a bag, or by other means. At this point, the organ will be managed in a standardized manner for transplantation/cell isolation.

Once the apparatus 2 is not being used, it is powered off at step S1020 by pressing the standby button 80a.

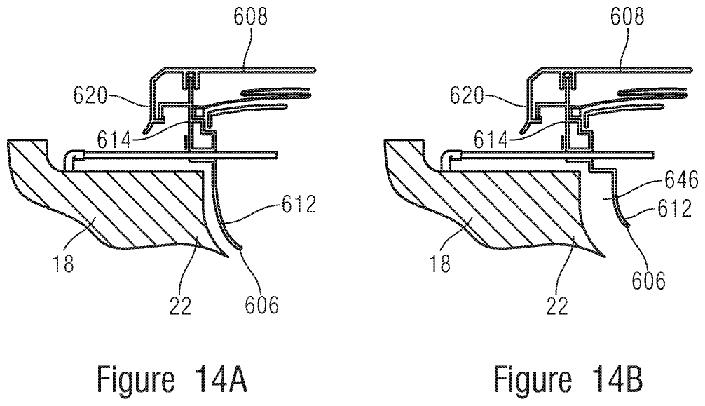

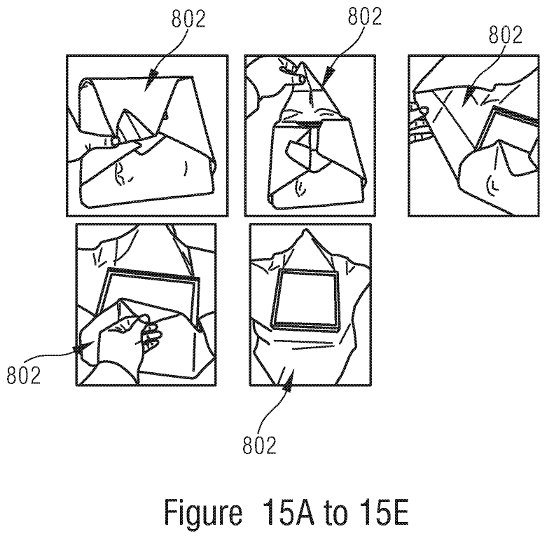

FIGS. 14A and 14B illustrate how the lower portion 612 of the storing portion 606 may be configured in accordance with the organ or body tissue type for which is to be used. For example, the lower portion 612 shown in FIG. 14A is relatively large in capacity, whereas the lower portion 612 shown in FIG. 14B is relatively small in capacity. The upper portion 614 of the storing portion 606 of both examples are, however, the same and are configured to engage with the docking portion 22 of the base unit 4. Furthermore, the space between the lower portion 612 of the arrangement shown in FIG. 14B may be filled with a packing material 646 in order to accommodate a smaller capacity lower portion 612. Thus, a bespoke container unit 6 can be configured/selected for a specific type of organ/body tissue and used within the same base unit 4. Each container unit is thus to some extent bespoke to a particular type of organ or body tissue portion. Container units can optionally be colour coded or visibly marked in some way to help a health care professional select a required container unit. For example red container units for ears, green for livers, purple for kidneys white for hands and yellow for face parts.

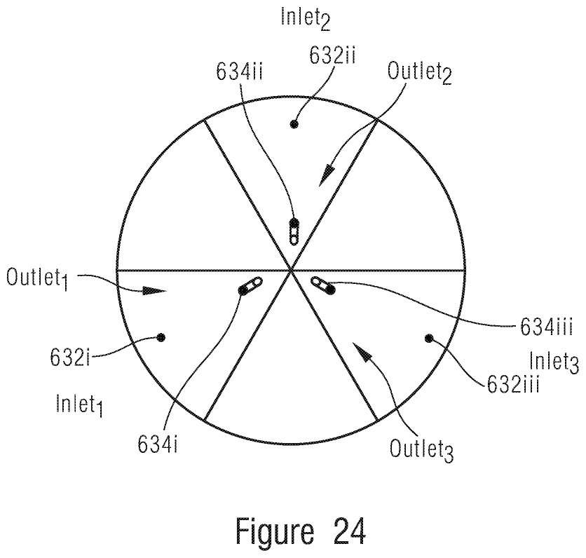

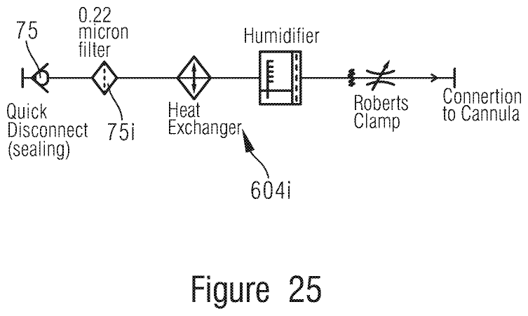

Each respective container unit not only has a respective internal dimensions/shape but also a requisite or ideal number of outlets in desired locations around the circumference of the container unit space. For example, as shown in FIG. 24, a container unit may have three separate outlets 634i, 634ii, 634iii spaced around the circumference of the container unit space. The outlets may be positioned to be aligned with a vessel or an organ or body tissue having a predetermined arrangement within the container unit to aid connection between of a cannula arrangement connected to the outlet to the correct vessel.

Each outlet 634i, 634ii, 634iii may be connected to a respective inlet 632i, 632ii, 632iii which is connected, in turn, to one of the flow lines of the base unit. A schematic representation of certain components of the container unit which are connected between each of the outlets 634i, 634ii, 634iii and respective inlets 632i, 632ii, 632iii is shown in FIG. 24. The components are shown for only one flow line and comprise a quick disconnect 75, 0.22 micron filter 75i, fluid processing device 604i (which comprises a heat exchanger and humidifier which are illustrated as separate components) a Roberts clamp and a connector for connecting to a cannula. Thus, a flow of persufflating gas can be supplied along each flow line at a specific flow rate and pressure which is suitable for the vessel or portion of the organ/body tissue which it supplies. This helps a surgeon efficiently connect lumens providing persufflation gas to desired respective regions of any organ or body tissue portion. Since container units may be of different size according to use a packer member or multiple packer members can be located between opposed surfaces of the container unit and the base unit.

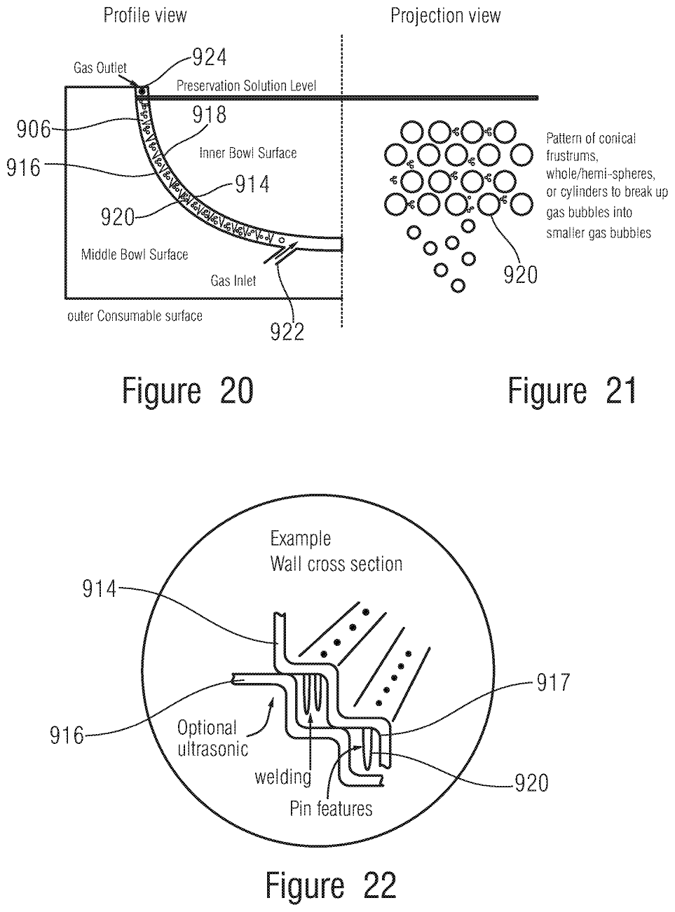

FIG. 20 shows an example in which fluid processing device is formed integrally with a lower portion 912 of a storing region 906 of a container unit. The lower portion 912 comprises an inner wall 914 and an outer wall 916 between which is defined an internal flow passageway 918 having baffles 920, in the form of cylindrical, conical or hemi-spherical formations, as shown in FIG. 21, within it. The internal flow passageway has a fluid inlet 922 at the lower end though which the persufflating fluid is introduced and a fluid outlet 924 at the upper end. FIG. 22 shows a variation of the example shown in FIGS. 20 and 21 in which the inner wall 914 and the outer wall 916 are stepped and are joined together at the corners, for example by ultrasonic welding, to form flow channels 917 extending circumferentially between the walls 914, 916. The flow channels 917 may be in fluid communication with each other. The baffles 920 are formed by pin features which are formed integrally with the upper wall 914 and extend downwardly within the flow channels 917.

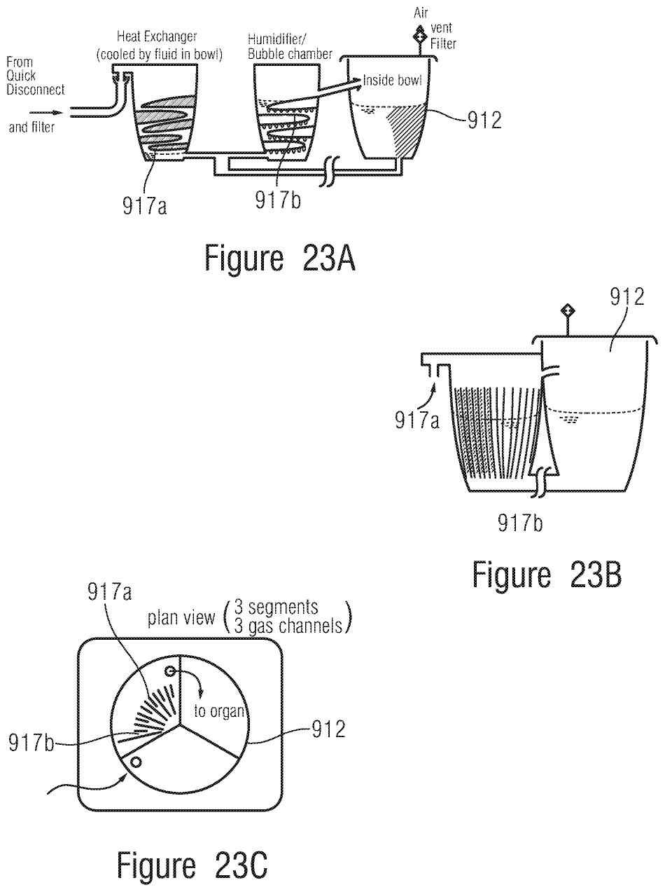

FIG. 23A shows a variation of the example shown in FIGS. 20 and 21 in which heat exchange is conducted in a first set of channels 917a and humidification is conducted in a second set of channels 917b independently of each other. FIG. 23A does not show the actual layout, but is intended to illustrate how certain components of the container unit are connected together. Both the first set of channels 917a and the second set of channels 917b are provided in fluid communication with bowl-like storage region of the container unit and are arranged in a fluted formation as shown in FIG. 23B. The first and second sets of channel 917a, 917b may be provided in a segment of the container unit only, as shown in FIG. 23C, and may be provided with an inlet port and an outlet port as described above. The arrangement may be replicated in other segments of the container unit for other flow channels used to persufflate and organ or body tissue. In this example, the heat exchange takes place before humidification which helps avoid a build up of condensation in the system upstream of the humidifier.

Each container unit can provide one, two, three or more persufflation gas outlets connectable via a respective lumen to a respective location of a specific organ or body tissue portion. Each outlet is supplied with gas having tailored qualities specific to the location on the organ or body tissue portion to which that gas is to be supplied. Thus each channel is independently parameter controllable. For example each channel can have a different (or optionally the same) temperature and/or pressure and/or flow rate and/or humidity and/or composition. These can be varied over time to fit a desired parameter profile. This is convenient when different regions of an organ or body tissue portion have different physical characteristics (such as density, porosity or granularity or the like).

Aptly the retained unit or base unit (e.g. the storage apparatus) is usage neutral whilst the disposable unit is organ type or body tissue portion type specific.

It will be appreciated that the container unit could be configured for use with the base unit 4 described in the previous examples.

It is anticipated that certain examples of the apparatus may be suitable for multiple organ/partial organ/composite tissue types (e.g. pancreas, kidney, liver, heart, bowel, lung, hand, arm, foot, leg, face, skin, etc.); anterograde or retrograde; for transplantation or cell isolation or other R&D applications. In particular, it is anticipated that certain examples of the apparatus may be suitable for anterograde persufflation of a pancreas for islet isolation.

Dedicated gas out-feed lines may be provided for exhausting gas directly through cannulas inserted into the organ or body tissue.

As described above, primary control is flow rate, secondary control is pressure, lines can be managed/controlled in series or parallel, progressively or all at same time; in an example, all lines managed at same time, in parallel, and any lines can be toggled between ‘active’ and inactive’ automatically or manually (for example, if an apparent leak, then automatically closed after a period to prevent ongoing gas wastage, but user can resolve the leak and switch the line ‘active’ again without affecting other lines).

Separate leak modes and maintenance (i.e. in-use) modes may be provided. Alternatively, they can be combined into a single mode.

Possible modes of operation include:

Disposable tubing not connected; in envisaged example, when first ‘started’, gas will come out this port, and control system might alert user to this as a leak or similar, but after a time of no improvement might make the line ‘inactive’ and close it off (principally to stop gas wastage).

Disposable tubing connected, but (Roberts) clamp closes the line; in envisaged example, this will restrict all gas flow in this line, and the control system will increase pressure to line (in case just a cannulation blockage/poor vascularisation), but after a time of no improvement might make the line ‘inactive’.

Disposable tubing connected, (Roberts) clamp open, but tubing not used to cannulate organ port/vessel; in envisaged example, when first ‘started’, gas will come out this port, and control system might alert user to this as a leak or similar, but after a time of no improvement might make the line ‘inactive’ and close it off (principally to stop gas wastage).

Disposable tubing connected, (Roberts) clamp open, organ poorly cannulated, which might exhibit as either a leak or a restriction—leak or restriction modes as described above may be implemented.

Disposable tubing connected, (Roberts) clamp open, organ suitably cannulated; in envisaged example, under control system, pressure gradually increased until target flow achieved, then pressure regulated to maintain target flow.

Table 1 below illustrated possible conditions and operation modes:

TABLE 1

| | Pressure lower than expected | Pressure OK | Pressure higher than expect |
|---|---|---|---|
| Flow | Potential scenarios: | Potential scenarios: | Potential scenarios: |
| too | Tubing line open, but not used in | Slight leak in | Control system not yet |
| high | cannulation | cannulation | reached target |
| | Leak in cannulation | Therefore: | Therefore: |
| | Therefore: | Alert user to ‘High | Maintain and manage |
| | Alert user to ‘High Flow’ via LED | Flow via LED and | under autoregulation |
| | and remote interface | remote interface | control algorithm |
| | After 30 mins, switch off line to | Maintain and manage | |
| | preserve gas, but LED stays lit | under autoregulation | |
| | User can restart at any time, which | control algorithm | |
| | would switch of the LED for a short | | |
| | period (but it will light again if | | |
| | situation remains) | | |
| Flow | Potential scenarios: | Potential scenarios: | Potential scenarios: |
| OK | Slightly atypical organ, but | Everything is OK | Slightly atypical organ, but |
| | everything is OK | Therefore: | everything is OK |
| | Therefore: | Maintain and manage | Therefore: |
| | Maintain and manage under | under autoregulation | Maintain and manage |
| | autoregulation control algorithm | control algorithm | underautoregulation |
| | | | control algorithm |
| Flow | Potential scenarios: | Potential scenarios: | Potential scenarios: |
| too | Control system not yet reached | Tubing line clamped | Tubing line clamped |
| low | target | Poor vascularisation | Poor vascularisation |
| | Therefore: | Therefore: | Therefore: |
| | Maintain and manage under | Alert user to ‘Low | Alert user to ‘Low Flow’ |
| | autoregulation control algorithm | Flow’ via LED and | via LED and remote |
| | | remote interface | interface |
| | | Maintain and manage | Maintain and manage |
| | | under autoregulation | under autoregulation |
| | | control algorithm | control algorithm |
| | | No point switching off | No point switching off as |
| | | as not wasting any | not wasting any gas within |
| | | gas | autoregulation parameters |

Preferably, the apparatus 2 should weigh less than 20 kg in total when in use.

The retained unit consists of majority of high value, high cost, reusable components and structural integrity to support the container unit (e.g. the Primary Organ Container).

The retained unit enables users/retrieval team to identify which organ they wish to persufflate (e.g. pancreas) and in which manner (e.g. anterograde), and provides ongoing monitoring and feedback of the gas in-feed lines and critical functions on the system; in one example, only critical control and feedback is via retained unit (e.g. which organ and which manner (or if under remote PC control for R&D), low pressure or low flow in individual lines, low battery, low pressure in gas source, temperature excursions), and specific values and historical charts are displayed through a remote monitoring connection (e.g. smartphone or tablet, via Bluetooth (RTM) broadcast, but no control).

The Primary Organ Container directly contains the organ and support media, could be single- use or multi-use (cleanable) or refurbishable (e.g. cleanable and replace gas lines), for one type of organ/tissue or multiple types; in preferred example, it will be to be sterile and disposable after use, principally moulded from a low cost plastic, to minimise costs and ensure clean and sterile.

The Primary Organ Container may a sealed container (with passive air vents) to prevent pressure build up during persufflation. To prevent contamination of organ/preservation media, all gas passages in and out of container (whether used or un-used) will have a filter for filtering out pathogens (e.g. to inhibit infection), such as a 0.22 micron filter on them, or a more restrictive filter, such as a 0.21 micron filter.

The Primary Organ Container may be configured to be suitable for only one organ type, some might be suitable for multiple organ types; in preferred example, focus is to minimise the amount of excess ‘free’ volume inside container, as this needs to be filled with a costly fluid, but external form will be consistent across all container types, to minimise free space in sealed retainer unit (as per illustrations already provided).

The Primary Organ Container may be supplied with recommended cannula, might be exact number of same size, or different interchangeable sizes according to organ variation; in preferred example, users will be able to select cannula that they think are most appropriate for organ case, including using their own preferred cannula which may not be supplied with the POC.

Cannula can connect to gas in-feed lines either above or below the fluid level; in one example, likely to be above the fluid level, which should ensure most consistent with current surgical practice.

Cannula ports can be in a regular or irregular pattern (according to organ-specific anatomical requirements), in any direction, depending on organ type; in envisaged example, likely to be an irregular pattern for pancreas.

Cannulas can stiff or flexible without occlusion; in envisaged example, likely to be flexible without occlusion.

In the secondary usage case where a secondary surgical drape is added, the adhesive region could have a tape which can be peeled off and would be placed onto an inner lip inside the organ container. In this case, the drape could either be peeled off if a soft adhesive is used or a similar perforation pattern could be implemented with repeated uses applied atop one another. If a bigger drape is needed the individual points could be folded atop one another ala an accordion. If required fold lines could be extrapolated for this; though the order of the folds is key to achieve the desired result.

Examples of modes of operation of a device in accordance with, or a variant of, the apparatus 2 described above are as follows:

An organ is attached to multiple channels with a defined flow rate maintained to each channel for the duration of leak-testing and subsequent persufflation. Flow will be directionally physiological (ie entering from and artery/portal vein and exiting a vein). Pressure thresholds will be tissue-specific but the general technique should be quite similar. A variety of parameters should be semi-continuously monitored throughout the duration of persufflation such that an end-user can monitor them remotely and a record of the procedure can be offloaded upon completion for patient records. These parameters should include but not be limited to: temperature, pressure to each line, flow rate to each line, recording of any alarms/overrides.

### 1 Turn on the Device

The device is activated once the consumable (e.g. container unit) is inserted, e.g. the user should activate the device by pressing a switch.

### 2. Define a Usage Case

The type of organ to be perfused should be inputted to define a set of parameters for the device to operate under. These parameters/thresholds may vary for each organ or tissue to be persufflated. This may come via a user inputted code, menu, hard switch, by something internal to the consumable to avoid user error, or by some other means.

### 3. Perform System Check

The system should perform a pre-use screen of all key system parameters to ensure it is capable of performing a defined persufflation ‘run’. Critically, testing the remaining battery charge should be performed and if below a given threshold the user should be prompted. This may trigger basic maintenance such as plugging the device in/swapping a battery or performing other key basic maintenance steps to ensure the device is ready to use.

### 4. Define Channels in Use

As the device may have 4 channels to send flow to and not all will be in use for a given case, the user may need to define which channels will be utilized for the procedure. This will happen once all vessels have been cannulated but prior to beginning flow to the organ/tissue. This number can range from 1 to 3 with capability to ‘turn off’ any channels not in use. This could be achieved by a series of Y/N questions, by a hard switch, or by entering a special mode where the user can depress a button to activate a given channel. Once this is established, a defined series of steps should have to be taken to modify the channels in use such that channels are not inadvertently activated/deactivated during use.

### 5. Enter ‘Leak-testing’ Mode

The device will now begin flowing gas to each of the channels defined above. Pressures will slowly ramp up according to a feedback loop defined below:

### 5.1 Gradually Ramp Pressures to each Channel

The pressure/flow to each channel will slowly ramp up according to a pre-defined profile.

### 5.2 Restrict pressure increase once a target flow rate has been achieved

Once a target flow rate has been achieved the pressure will be maintained for a given channel. This operates under a ‘flow-control’ paradigm where we are aiming for a target flow rate while trying to minimize the pressure to a flow path avoiding any unnecessary barro-trauma. This flow rate should be continuously maintained for the rest of the procedure until the organ is disconnected or an upper pressure threshold has been reached (see below). In the case of anterograde persufflation of the pancreas we may define this target as 25 ml/min of gas flow.

### 5.3 Activate a ‘User-alarm’ if a Channel Pressure Does Not Reach a Specified Threshold (Low)

If the pressure does not meet a defined threshold to a given channel, the flow to the channel should be capped and the presence of a ‘possible leak’ should be indicated to the user. This may come in the form of a light, screen cue audible noise or other alarm. For the pancreas, this would typically occur below 10-15 mmHg but occasionally stable flows could be achieved in the 12-15 mmHg range with no observable leaks found. Therefore perhaps there could be a coded statement to this alarm (ie red for very low and yellow for low) and a user override could be built into the system should the pressure not meet this threshold in the absence of observable leaks.

### 5.4 Activate a ‘User-alarm’ if a Channel Pressure Exceeds a Specified Threshold

If the pressure exceeds a defined threshold to a given channel, the pressure to the channel should be capped and a user alarm initiated. This should inform the user to check for an obstruction to flow through the organ or tissue. This may typically include: kinks in the tubing, twists in the vessel, or if the cannulae is somehow jammed into the tissue or vessel wall. Once the user has inspected the designated line, there should be an override function to expand the defined threshold beyond that typical for the usage case. Once a secondary upper limit has been reached, a secondary user override should enable further pressure increase to the maximum acceptable pressure for a designated usage case.

For example, in the pancreas we would expect a pressure between 15-25 mmHg to any given vascular bed. In some cases, particularly poorly flushed tissue such as that obtained following a Donation after Circulatory Death (DCD) procurement, the pressure may exceed this threshold. If this is the case we would inspect for obstructions to flow and then increase pressures to a 50 mmHg threshold. If this is reached and there are still no signs of occlusion, we may then continue to increase the pressure to an upper limit of 80 mmHg.

### 5.5 Exit Leak-testing Mode And Enter ‘Maintenance Mode’

Once all active channels meet predetermined guidelines and the user is happy there are no large leaks, the user shall exit the ‘leak testing mode’ and proceed to maintenance mode. This will restrict the capacity to alter active channels and aim to maintain the specified flow to each channel in use.

### 6. Maintenance Mode

Should the pressure dramatically increase to maintain a given flow >10 mmHg from entering maintenance mode, an alarm should be triggered indicating an obstruction to flow.

If facilities are available, the user should then have the capacity to re-enter ‘leak-testing mode’ to fix the problem.

Typically, as the vascular bed relaxes and is cleared of liquid, the pressure required to maintain a given flow rate drops 5-10 mmHg over the first 30-60 min of persufflation. This has been observed across multiple organs/tissues and should be accounted for such that the flow is limited to the target value; minimizing the opportunity for barrotrauma.

### ‘Series A-PSF’

An organ is attached to multiple channels with a defined flow rate maintained to each channel for the duration of leak-testing and subsequent persufflation. Flow will be directionally physiological (ie entering from and artery/portal vein and exiting a vein). Pressure thresholds will be tissue-specific but the general technique should be quite similar. A variety of parameters should be semi-continuously monitored throughout the duration of persufflation such that an end-user can monitor them remotely and a record of the procedure can be offloaded upon completion for patient records. These parameters should include but not be limited to: temperature, pressure to each line, flow rate to each line, recording of any alarms/overrides.

### 1 Turn on the Device

The user should activate the device by pressing a switch.

### 2. Define a Usage Case

The type of organ to be perfused should be inputted to define a set of parameters for the device to operate under. These parameters/thresholds may vary for each organ or tissue to be persufflated. This may come via a user inputted code, menu, hard switch, by something internal to the consumable to avoid user error, or by some other means.

### 3. Perform System Check

The system should perform a pre-use screen of all key system parameters to ensure it is capable of performing a defined persufflation ‘run’. Critically, testing the remaining battery charge should be performed and if below a given threshold the user should be prompted. This may trigger basic maintenance such as plugging the device in/swapping a battery or performing other key basic maintenance steps to ensure the device is ready to use.

### 4. Define Channels in Use

As the device will have 4 channels to send flow to and not all will be in use for a given case, the user will need to define which channels will be utilized for the procedure. This will happen once all vessels have been cannulated but prior to beginning flow to the organ/tissue. This number can range from 1 to 3 with capability to ‘turn off’ any channels not in use. This could be achieved by a series of Y/N questions, by a hard switch, or by entering a special mode where the user can depress a button to activate a given channel. Once this is established, a defined series of steps should have to be taken to modify the channels in use such that channels are not inadvertently activated/deactivated during use.

### 5. Enter ‘Leak-testing’ Mode

The device will now begin flowing gas to each of the channels defined above. Pressures will slowly ramp up according to a feedback loop defined below:

### 5.1 Gradually Ramp Pressures to the Selected Channel

The pressure/flow to each channel will slowly ramp up according to a pre-defined profile.

### 5.2 Restrict Pressure Increase Once a Target Flow Rate has been Achieved

Once a target flow rate has been achieved the pressure will be maintained to the channel. This operates under a ‘flow-control’ paradigm where we are aiming for a target flow rate while trying to minimize the pressure to a flow path avoiding any unnecessary barro-trauma. This flow rate should be continuously maintained until testing of this channel has been completed an upper pressure threshold has been reached (see below). In the case of the pancreas we have defined this target as 25 ml/min of gas flow.

### 5.3 Activate a ‘User-alarm’ if a Channel Pressure Does Not Reach a Specified Threshold (Low)

If the pressure does not meet a defined threshold, the presence of a ‘possible leak’ should be indicated to the user. This may come in the form of a light, screen cue audible noise or other alarm. For the pancreas, this would typically occur below 10-15 mmHg but occasionally stable flows could be achieved in the 12-15 mmHg range with no observable leaks found. Therefore perhaps there could be a coded statement to this alarm (ie red for very low and yellow for low) and a user override could be built into the system should the pressure not meet this threshold in the absence of observable leaks. Alarm may also be embodied by a low frequency audible noise (beep).

### 5.4 Activate a ‘User-alarm’ if a Channel Pressure Exceeds a Specified Threshold

If the pressure exceeds a defined, the pressure to the channel should be capped and a user alarm initiated. This should inform the user to check for an obstruction to flow through the organ or tissue. This may typically include: kinks in the tubing, twists in the vessel, or if the cannulae is somehow jammed into the tissue or vessel wall. Once the user has inspected the line, there should be an override function to expand the defined threshold beyond that typical for the usage case. Once a secondary upper limit has been reached, a secondary user override should enable further pressure increase to the maximum acceptable pressure for a designated usage case. Alarm may also be embodied by a high frequency audible noise (beep).

For example, in the pancreas we would expect a pressure between 15-25 mmHg to any given vascular bed. In some cases, particularly poorly flushed tissue such as that obtained following a DCD procurement, the pressure may exceed this threshold. If this is the case we would inspect for obstructions to flow and then increase pressures to a 50 mmHg threshold. If this is reached and there are still no signs of occlusion, we may then continue to increase the pressure to an upper limit of 80 mmHg.

### 5.5 Proceed to Inspection of the Next Channel

Once the user is happy with a channel, the flows to this channel can be stopped and the process repeated (steps 5.1-5.5) for the next channel. If there are no further channels to leak-test, the user will indicate this to the device and flow will be restored to all channels.

### 5.6 Exit Leak-testing Mode and Enter ‘Maintenance Mode’

Once all active channels meet predetermined guidelines and the user is happy there are no large leaks, the user shall exit the ‘leak testing mode’ and proceed to maintenance mode. This will restrict the capacity to alter active channels and aim to maintain the specified flow to each channel in use.

### 6. Maintenance Mode

Should a sharp decline in pressure to any channel occur; such as greater than 15 mmHg over any 3 min period, an alarm should be triggered to inspect the line with a timestamp of when this occurred. Likewise, should the pressure dramatically increase to maintain a given flow >10 mmHg from entering maintenance mode, an alarm should be triggered indicating an obstruction to flow.

If facilities are available, the user should then have the capacity to re-enter ‘leak-testing mode’ to fix the problem.

Typically, as the vascular bed relaxes and is cleared of liquid, the pressure required to maintain a given flow rate drops 5-10 mmHg over the first 30-60 min of persufflation. This has been observed across multiple organs/tissues and should be accounted for such that the flow is limited to the target value; minimizing the opportunity for barrotrauma.

### ‘R-PSF’

For this case, flow will run directionally opposite of physiological blood flow. For example, flow may enter from a vein and exit either through the artery or through a series of ‘pin pricks’ manually placed into the organ to act as a drainage shunt. Pressure thresholds will be tissue-specific but the general technique should be quite similar. A variety of parameters should be semi-continuously monitored throughout the duration of persufflation such that an end-user can monitor them remotely and a record of the procedure can be offloaded upon completion for patient records. These parameters should include but not be limited to: temperature, pressure to each line, flow rate to each line, recording of any alarms/overrides.

### 1 Turn on the Device

The user should activate the device by pressing a switch.

### 2. Define a Usage Case

The type of organ to be perfused should be inputted to define a set of parameters for the device to operate under. These parameters/thresholds may vary for each organ or tissue to be persufflated. This may come via a user inputted code, menu, hard switch, by something internal to the consumable to avoid user error, or by some other means.

### 3. Perform System Check

The system should perform a pre-use screen of all key system parameters to ensure it is capable of performing a defined persufflation ‘run’. Critically, testing the remaining battery charge should be performed and if below a given threshold the user should be prompted. This may trigger basic maintenance such as plugging the device in/swapping a battery or performing other key basic maintenance steps to ensure the device is ready to use.

### 4. Define Channels in Use

As the device will have 4 channels to send flow to and not all will be in use for a given case, the user will need to define which channels will be utilized for the procedure. This will happen once all vessels have been cannulated but prior to beginning flow to the organ/tissue. This number can range from 1 to 3 with capability to ‘turn off’ any channels not in use. This could be achieved by a series of Y/N questions, by a hard switch, or by entering a special mode where the user can depress a button to activate a given channel. Once this is established, a defined series of steps should have to be taken to modify the channels in use such that channels are not inadvertently activated/deactivated during use.

### 5. Maintenance Mode

As R-PSF operates on a ‘pressure controlled’ paradigm, the system will need to supply a constant pre-defined pressure to a given channel for the duration of the procedure. For example, for the liver this may be set to 18 mmHg. Flow parameters should be monitored and recorded until cessation of persufflation.

In the drawings like reference numerals refer to like parts. It will be appreciated from the discussion above that the embodiments shown in the figures are merely exemplary, and include features which may be generalised, removed or replaced as described herein and as set out in the claims. With reference to the drawings in general, it will be appreciated that schematic functional block diagrams are used to indicate functionality of systems and apparatus described herein. In addition the processing functionality may also be provided by devices which are supported by an electronic device. It will be appreciated however that the functionality need not be divided in this way, and should not be taken to imply any particular structure of hardware other than that described and claimed below. The function of one or more of the elements shown in the drawings may be further subdivided, and/or distributed throughout apparatus of the disclosure. In some embodiments the function of one or more elements shown in the drawings may be integrated into a single functional unit.

As will be appreciated by the skilled reader in the context of the present disclosure, each of the examples described herein may be implemented in a variety of different ways. Any feature of any aspects of the disclosure may be combined with any of the other aspects of the disclosure. For example method aspects may be combined with apparatus aspects, and features described with reference to the operation of particular elements of apparatus may be provided in methods which do not use those particular types of apparatus. In addition, each of the features of each of the embodiments is intended to be separable from the features which it is described in combination with, unless it is expressly stated that some other feature is essential to its operation. Each of these separable features may of course be combined with any of the other features of the embodiment in which it is described, or with any of the other features or combination of features of any of the other embodiments described herein. Furthermore, equivalents and modifications not described above may also be employed without departing from the invention.

Certain features of the methods described herein may be implemented in hardware, and one or more functions of the apparatus may be implemented in method steps. It will also be appreciated in the context of the present disclosure that the methods described herein need not be performed in the order in which they are described, nor necessarily in the order in which they are depicted in the drawings. Accordingly, aspects of the disclosure which are described with reference to products or apparatus are also intended to be implemented as methods and vice versa. The methods described herein may be implemented in computer programs, or in hardware or in any combination thereof. Computer programs include software, middleware, firmware, and any combination thereof. Such programs may be provided as signals or network messages and may be recorded on computer readable media such as tangible computer readable media which may store the computer programs in non-transitory form. Hardware includes computers, handheld devices, programmable processors, general purpose processors, application specific integrated circuits (ASICs), field programmable gate arrays (FPGAs), and arrays of logic gates. Other examples and variations of the disclosure will be apparent to the skilled addressee in the context of the present disclosure.

1. An apparatus for storing an organ or body tissue, the apparatus comprising:
a container unit comprising a container body defining a storage region for storing an organ or body tissue, wherein the container body includes an opening to enable the organ or body tissue to be admitted to, and removed from, the storage region through the opening; and
a surgical drape comprising: (i) a securing portion which is securable to the container unit, (ii) a peripheral outer portion which surrounds the securing portion, and (iii) a frangible portion separating the securing portion from the peripheral outer portion;
wherein the securing portion is securable to the container body to enable the drape to extend around a portion of the periphery of the opening with the peripheral outer portion of the drape arranged outward of the opening; and
wherein the frangible portion extends around the securing portion to enable the peripheral outer portion to be separated from the securing portion by tearing the frangible portion.

2. The apparatus of claim 1, wherein the container unit has an attachment portion to which the surgical drape is securable.

3. The apparatus of claim 2, wherein the attachment portion surrounds at least a portion of the opening of the container unit.

4. The apparatus of claim 1, wherein the securing portion of the drape comprises an adhesive region for securing the drape to the container unit.

5. The apparatus of claim 1, wherein the drape includes an access opening which corresponds to the opening of the container unit to enable surgical drape to be secured to the container unit with the access opening in alignment with the opening of the container unit.

6. The apparatus of claim 4, wherein the drape includes an access opening which corresponds to the opening of the container unit to enable surgical drape to be secured to the container unit with the access opening in alignment with the opening of the container unit, and wherein the adhesive region surrounds at least a portion of the access opening.

7. The apparatus of claim 1, wherein the surgical drape comprises a first surgical drape, and the apparatus further comprises a second surgical drape; and
wherein the second surgical drape comprises a second securing portion which is securable to the container body to enable the second drape to extend around a portion of the periphery of the opening.

8. The apparatus of claim 7, wherein the container unit comprises a second attachment portion to which the second surgical drape is securable.

9. The apparatus of claim 8, wherein the second attachment portion comprises an inner surface of a wall of the container body.

10. The apparatus of claim 1, wherein the container unit further comprises an inner lid member which is configured to be secured to the container body so as to enclose the organ or body tissue within the storage region between the container body and the inner lid member.

11. The apparatus of claim 9, wherein the container unit further comprises an inner lid member which is configured to be secured to the container body so as to enclose the organ or body tissue within the storage region between the container body and the inner lid member, and wherein the second attachment portion is an inner surface of a wall of a container unit which is external the storage region enclosed by the lid member and the container body.

12. The apparatus of claim 7, wherein the container unit further comprises an inner lid member which is configured to be secured to the container body so as to enclose the organ or body tissue within the storage region between the container body and the inner lid member, and wherein the apparatus is arranged to enable the second drape to be secured to the container body so that a portion of the second drape overlies the inner lid member.

13. The apparatus of claim 10, wherein the container unit comprises an outer lid member which is configured to be secured to the container body so as to enclose the inner lid member between the container body and the outer lid member.

14. A surgical drape configured to be secured to a container unit for storing an organ or body tissue, the surgical drape comprising:
an access opening arranged to enable an organ or body tissue to be passed through the access opening;
a securing portion surrounding the access opening which is securable to a securing portion of a said container unit;
a peripheral outer portion which surrounds the securing portion; and
a frangible portion separating the securing portion from the peripheral outer portion;
wherein the securing portion is securable to the container body to: (i) enable the access opening of the drape to align with an opening of the container body, (ii) enable the drape to extend around a portion of the periphery of the opening of the container unit with the peripheral outer portion of the drape arranged outward of the opening; and
wherein the frangible portion extends around the securing portion to enable the peripheral outer portion to be separated from the securing portion by tearing the frangible portion.

15. The apparatus of claim 14, wherein the securing portion of the drape comprises an adhesive region for securing the drape to the container unit.

16. An apparatus for preserving an organ or body tissue, the apparatus comprising:
a storage apparatus comprising: (i) a fluid supply system comprising a store of persufflation fluid to be supplied to a said organ or body tissue, and (ii) a container unit receiving portion;
a container unit removably insertable into the container unit receiving portion, wherein the container unit comprises a container body defining a storage region for storing an organ or body tissue, wherein the container body includes an opening to enable the organ or body tissue to be admitted to, and removed from, the storage region through the opening; and
a surgical drape comprising: (i) a securing portion which is securable to the container unit, (ii) a peripheral outer portion which surrounds the securing portion, and (iii) a frangible portion separating the securing portion from the peripheral outer portion;
wherein the securing portion of the drape is securable to the container body to enable the drape to extend around a portion of the periphery of the opening with the peripheral outer portion of the drape arranged outward of the opening to protect a portion of a surface of the storage apparatus; and
wherein the frangible portion extends around the securing portion to enable the peripheral outer portion to be separated from the securing portion by tearing the frangible portion.

17. The apparatus of claim 16, wherein the portion of the surface of the storage apparatus comprises a portion of a periphery of the container unit receiving portion.

18.-28. (canceled)
